(19) United States
(12) Patent Application Publication
(10) Pub. No.: US 2026/0250469 A1
(43) Pub. Date: Aug. 27, 2026

(54) PROCESSES FOR MAKING THREE-DIMENSIONAL POLYMER NETWORKS

(71) Applicant: SAFEGUARD DX LTD, St. Albans (GB)

(72) Inventors: Holger KLAPPROTH, Freiburg (DE); Sonja BEDNAR, Gundelfingen (DE)

(73) Assignee: SAFEGUARD DX LTD, St. Albans (GB)

(21) Appl. No.: 18/878,206

(22) PCT Filed: Jun. 27, 2023
(86) PCT No.: PCT/EP2023/067415

§ 371 Date: Dec. 23, 2024

Related U.S. Application Data
(60) Provisional application No. 63/356,171, filed on Jun. 28, 2022.

(51) Int. Cl. C08J 3/075; C08J 3/24; C12Q 1/6837; C12Q 1/6876

(52) U.S. Cl. CPC C08J 3/075; C08J 3/24; C12Q 1/6837; C12Q 1/6876; C08J 2333/10; C08J 2333/26; C08J 2381/08

(57) ABSTRACT

This disclosure provides processes for making three-dimensional cross-linked polymer networks having transport channels, processes for making arrays comprising the three-dimensional networks, arrays comprising the three-dimensional networks, and uses of the three-dimensional networks and arrays.

### 1. CROSS-REFERENCE TO RELATED APPLICATIONS

This application claims the priority benefit of U.S. provisional application No. 63/356,171, filed Jun. 28, 2022, the contents of which are incorporated herein in their entireties by reference thereto.

### 2. BACKGROUND

WO 2017/103128 and WO 2018/234253, the contents of which are incorporated herein by reference in their entireties, describe three-dimensional polymer networks comprising cross-linked polymer chains and one or more transport channels. The transport channels permit molecules in solution, e.g., analyte molecules, to access the polymer molecules within the network. The polymer chains can be cross-linked to probe molecules, and the transport channels provide a greater surface area for binding of analytes to probe molecules.

The networks described in WO 2017/103128 and WO 2018/234253 allow for faster hybridization of a given amount of analyte than networks lacking transport channels because the transport channels can effectively increase the surface area of the network, exposing more probes to the sample in a given amount of time. Additionally, the networks can bind more analyte than the same volume of a transport channel-free network because the transport channels decrease or eliminate the problem whereby analyte or other components of a sample bound to probes at or near the surface of the network block access to probes located in the interior of the network. Another advantage of the networks of WO 2017/103128 and WO 2018/234253 is that the high amount of analyte loading made possible by the transport channels allows for a more sensitive detection of analyte than may be possible with a transport channel-free network, i.e., the signal to noise ratio can be improved compared to transport channel-free networks because a given amount of analyte can be concentrated in a smaller network volume. Yet another advantage of the networks of WO 2017/103128 and WO 2018/234253 is that the high analyte loading made possible by the transport channels allows for quantification of a wider range of analyte concentrations compared to transport channel-free networks.

WO 2017/103128 and WO 2018/234253 describe various processes for making three-dimensional polymer networks with transport channels. An exemplary process can include the following steps: (a) exposing a mixture comprising an aqueous salt solution, a polymer, and a cross-linker to salt crystal forming conditions, (b) exposing the mixture to cross-linking conditions to cross-link the polymer to form a cross-linked polymer network, and (c) contacting the cross-linked polymer network with a solvent to dissolve the salt crystals and form one or more channels.

### 3. SUMMARY

This disclosure provides new processes for making three-dimensional networks having one or more transport channels. The present disclosure is based, in part, on the surprising discovery that the homogeneity of three-dimensional networks and arrays comprising them can be improved by using a relatively low concentration of polymer chains and/or salt when making the networks. The present disclosure is also based, in part, on the surprising discovery that using a relatively low cross-linking energy for cross-linking the polymer chains during production of the networks can also improve homogeneity of the networks and arrays.

It is believed that when relatively high concentrations of polymer chains and/or salt are used in the mixtures used to make three-dimensional networks of the type described in WO 2017/103128 and WO 2018/234253, components can prematurely precipitate from the mixture, decreasing homogeneity of arrays comprising the three-dimensional networks and negatively impacting their functionality. It is further believed that by lowering the concentration of polymer chains and/or salt, premature precipitation can be reduced or avoided, leading to improved homogeneity and functionality of the arrays. It is further believed that lowering the cross-linking energy can facilitate extraction of non-cross-linked material, leading to more homogeneous arrays.

Accordingly, in some aspects, the disclosure provides processes for making three-dimensional networks in which the concentration of polymer and/or salt is/are lower than the concentrations of polymer and/or salt described in WO 2017/103128 and WO 2018/234253, and/or in which the cross-linking energy is lower than the cross-linking energy described in WO 2017/103128 and WO 2018/234253. Exemplary processes for making three-dimensional networks are described in Section 5.1 and specific embodiments 1 to 221, infra.

The disclosure also provides processes for making arrays comprising a plurality of three-dimensional hydrogel networks. Exemplary processes for making arrays are described in Section 5.1 and specific embodiments 222 to 230, infra.

This disclosure also provides three-dimensional networks made by the processes of the disclosure, pluralities of three-dimensional networks, and arrays comprising a plurality of the three-dimensional networks of the disclosure and a substrate. Exemplary three-dimensional networks, pluralities of three-dimensional networks, and arrays are described in Sections 5.2 and 5.3, and specific embodiments 231 to 296, infra.

This disclosure also provides processes for using the three-dimensional networks and arrays of the disclosure to detect and/or measure an analyte in a sample, preferably a liquid sample. Exemplary processes for using the three-dimensional networks and arrays are described in Sections 5.4 and 5.5, and specific embodiments 297 to 348, infra.

The disclosure also provides kits useful for making a three-dimensional network and/or array of the disclosure. Exemplary kits are described in Section 5.6 and specific embodiments 349 to 351, infra.

### 4. BRIEF DESCRIPTION OF THE FIGURES

FIG. 1A-1B show hybridization signals and coefficients of variation (% CV) for three-dimensional networks made using different concentrations of phosphate, polymer, and probe, and different cross-linking energies (Example 1). FIG. 1A shows results for probe AllCan1 and FIG. 1B shows results for probe Sau-71p.

FIG. 2 shows the print plan for three-dimensional networks of Example 2 made using different concentrations of phosphate, polymer, and probe, and different cross-linking energies. Polymer concentrations are shown along the left side of the figure, phosphate concentrations are shown along the top of the figure, and probe concentrations are shown along the bottom of the figure. Rows A and J included spatial control spots (cc) for orienting the plate.

FIGS. 3A-3B show Cy3 (FIG. 3A) and Cy5 (FIG. 3B) fluorescence images of the three-dimensional networks of Example 2.

FIGS. 4A-4B show Cy3 (FIG. 4A) and Cy5 (FIG. 4B) signal intensities for probe E. coli-1637p measured for three-dimensional networks of Example 2.

FIGS. 5A-5B show coefficients of variation (% CV) using Cy3 (FIG. 4A) and Cy5 (FIG. 4B) fluorescence for the three-dimensional networks of Example 2. Data shown is for probe E. coli-1637p.

FIGS. 6A-6B show Cy3 (FIG. 6A) and Cy5 (FIG. 6B) signal intensities for probe Entb-132p measured for three-dimensional networks of Example 2.

FIGS. 7A-7B show coefficients of variation (% CV) using Cy3 (FIG. 7A) and Cy5 (FIG. 7B) fluorescence for the three-dimensional networks of Example 2. Data shown is for probe Ent-132p.

### 5. DETAILED DESCRIPTION

### 5.1. Processes For Making Three-Dimensional Polymer Networks and Arrays

In one aspect, the processes of the disclosure for making three-dimensional polymer networks comprise (a) exposing a mixture comprising an aqueous salt solution, a polymer, a cross-linker and, optionally, one or more probes to salt crystal forming conditions, (b) exposing the mixture to cross-linking conditions to cross-link the polymer for form a cross-linked polymer network, and (c) contacting the cross-linked polymer network with a solvent to dissolve the salt crystals and form one or more transport channels.

The concentration of the polymer chains in the mixture can be selected so that precipitation of the polymer chains from the mixture does not occur before formation of salt crystals (e.g., as observed by visual inspection, for example via a microscope or digital image(s)). For example, if precipitation of polymer is observed prior to formation of salt crystals during step (a), the concentration of polymer can be reduced until precipitation is no longer observed prior to formation of salt crystals. Alternatively, or in addition, the salt concentration can be adjusted to reduce or avoid premature precipitation of polymer. In some embodiments, concentrations of polymer and salt are selected so that polymer and salt co-precipitate during step (a).

The processes can further comprise a step of forming the mixture by combining an aqueous salt solution, a polymer, a cross-linker and, optionally, one or more probes, and/or further comprise a step of applying the mixture to a substrate (e.g., a substrate described in Section 5.3) prior to exposing the mixture to salt crystal forming conditions. If the polymer being used has a pre-attached cross-linker (e.g., when using a copolymer polymerized from a monomer comprising a cross-linker), the step of forming the mixture can comprise combining an aqueous salt solution with the polymer and, optionally, one or more probes.

The mixture can be applied to a substrate prior to exposing the mixture to salt crystal forming conditions for example, by spraying the mixture onto a surface of the substrate (e.g., at 1024 sites on the surface). The mixture can be applied to the surface using a DNA chip spotter or inkjet printer, for example. In a preferred embodiment, the mixture is sprayed using an inkjet printer. This permits a simple and rapid application of the mixture to a large number of spots on the substrate. The spots can be arranged, for example, in the form of a matrix in several rows and/or columns. Preferably, the salt content in the mixture during printing is below the solubility limit so that the mixture does not crystallize in the printing head of the printer. The volume of mixture applied at individual spots can be, for example, 100 pl, 200 pl, 300 pl, 400 pl, 500 pl, 750 pl, 1 nl, 2 nl, 3 nl, 4 nl, or 5 nl, or can be selected from a range bounded by any two of the foregoing values (e.g., 100 pl to 5 nl, 100 pl to 1 nl, 300 pl to 1 nl, 200 pl to 750 nl, 100 pl to 500 pl, 200 pl to 2 nl, 500 pl to 2 nl 1 nl to 2 nl, and so on and so forth). In preferred embodiments, the spot volume is 200 pl to 4 nl.

The diameter of the individual spots will depend on the composition of the mixture, the volume of the mixture applied, and the surface chemistry of the substrate. Spot diameters typically range between 80 μm to 1000 μm and can be obtained by varying the foregoing parameters. In various embodiments, the spot diameters are 80 μm, 100 μm, 120 μm, 140 μm, 160 μm, 180 μm, 200 μm, 300 μm, 400 μm, 500 μm, 600 μm, 700 μm, 800 μm, 900 μm, or 1000 μm, or selected from a range bounded by any two of the foregoing embodiments, e.g., 80 μm to 200 μm, 100 μm to 120 μm, 120 μm to 140 μm, 120 μm to 180 μm, 140 μm to 160 μm, 160 μm to 180 μm, 180 μm to 200 μm, 120 μm to 200 μm, 100 μm to 400 μm, 160 μm to 600 μm, or 120 μm to 700 μm, and so on and so forth. In a preferred embodiment, the diameter ranges from 100 μm to 200 μm or a subrange thereof.

Polymers, cross-linkers, salts, and probes that can be used to make the networks are described in Sections 5.1.1, 5.1.2, 5.1.3, and 5.1.4, respectively. Suitable salt crystal forming conditions are described in Section 5.1.5. Suitable cross-linking conditions are described in Section 5.1.6. Suitable solvents for dissolving the salt crystals are described in Section 5.1.7.

In some embodiments, the polymer used in the processes has at least one cross-linker group per polymer molecule. In a preferred embodiment, the polymer has at least two cross-linker groups per molecule. In a particularly preferred embodiment, the polymer has at least two photoreactive cross-linker groups per molecule. In these embodiments, separate polymer and cross-linker molecules are not needed.

### 5.1.1. Polymers

The three-dimensional networks of the disclosure can comprise a cross-linked homopolymer, copolymer, mixtures of homopolymers, mixtures of copolymers, or mixtures of one or more homopolymers and one or more copolymers. The term “polymer” as used herein includes both homopolymers and/or copolymers. The term “copolymer” as used herein includes polymers polymerized from two or more types of monomers (e.g., bipolymers, terpolymers, quaterpolymers, etc.). Copolymers include alternating copolymers, periodic copolymers, statistical copolymers, random copolymers, block copolymers, linear copolymers and branched copolymers. The three-dimensional networks of the disclosure can comprise any combination of the foregoing types of polymers. Reagents and methods for making such polymers are known in the art (see, e.g., Ravve, A., Principles of Polymer Chemistry, Springer Science+Business Media, 1995; Cowie, J. M. G., Polymers: Chemistry & Physics of Modern Materials, 2^{nd} Edition, Chapman & Hall, 1991; Chanda, M., Introduction to Polymer Science and Chemistry: A Problem-Solving Approach, 2^{nd} Edition, CRC Press, 2013; Nicholson, J. W., The Chemistry of Polymers, 4^{th} Edition, RSC Publishing, 2012; the contents of each of which are herein incorporated by reference in their entirety).

Preferred polymers are hydrophilic and/or contain hydrophilic groups. The polymer used in the processes of the disclosure is preferably water-soluble. Thus, in some embodiments, the polymer comprises water-soluble polymer chains. In an embodiment, the polymer is a copolymer that has been polymerized from two or more species of monomers selected to provide a desired level of water solubility. For example, water solubility of a copolymer can be controlled by varying the amount of a charged monomer, e.g., sodium 4-vinylsulfonate, used to make the copolymer.

When cross-linked, water-soluble polymers form water-swellable gels or hydrogels. Hydrogels absorb aqueous solutions through hydrogen bonding with water molecules. The total absorbency and swelling capacity of a hydrogel can be controlled by the type and degree of cross-linkers used to make the gel. Low cross-link density polymers generally have a higher absorbent capacity and swell to a larger degree than high cross-link density polymers, but the gel strength of high cross-link density polymers is firmer and can maintain network shape even under modest pressure.

A hydrogel's ability to absorb water is a factor of the ionic concentration of the aqueous solution. In certain embodiments, a hydrogel of the disclosure can absorb up to 50 times its weight (from 5 to 50 times its own volume) in deionized, distilled water and up to 30 times its weight (from 4 to 30 times its own volume) in saline. The reduced absorbency in saline is due to the presence of valence cations, which impede the polymer's ability to bond with the water molecule.

The three-dimensional network of the disclosure can comprise a copolymer that has been polymerized from one, two, thee, or more than three species of monomers, wherein one, two, three or more than three of the species of monomers have a polymerizable group independently selected from an acrylate group (e.g., acrylate, methacrylate, methyl methacrylate, hydroxyethyl methacrylate, ethyl acrylate, 2-phenyl acrylate), an acrylamide group (e.g., acrylamide, methacrylamide, dimethylacrylamide, ethylacrylamide), an itaconate group (e.g., itaconate, 4-methyl itaconate, dimethyl itaconate) and a styrene group (e.g. styrene, 4-methyl styrene, 4-ethoxystyrene). Preferred polymerizable groups are acrylate, methacrylate, ethacrylate, 2-phenyl acrylate, acrylamide, methacrylamide, itaconate, and styrene. In some embodiments, one of the monomers used to make the copolymer is charged, e.g., sodium 4-vinylbenzenesulfonate.

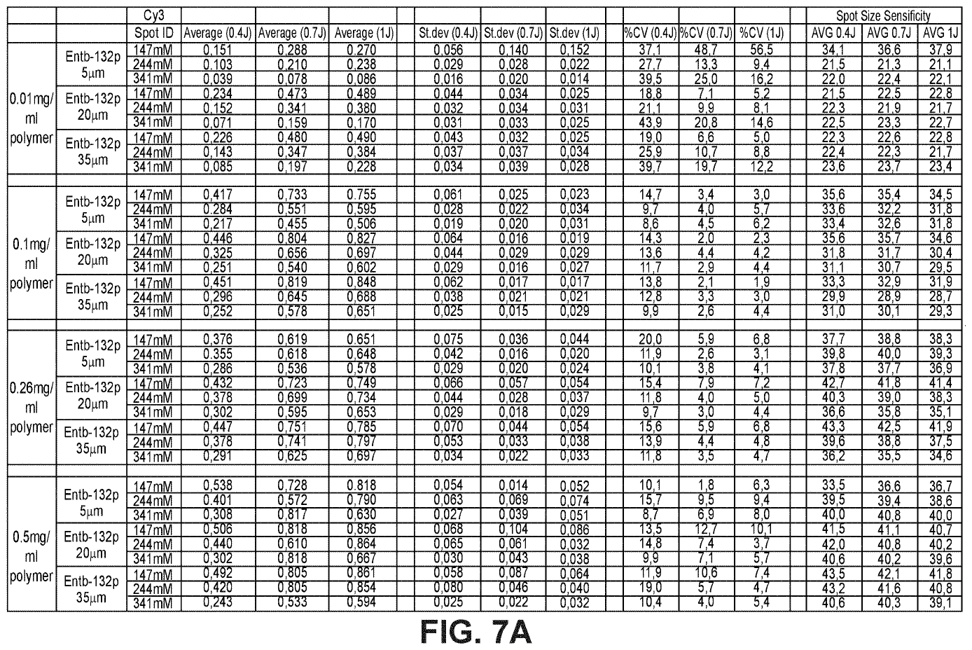

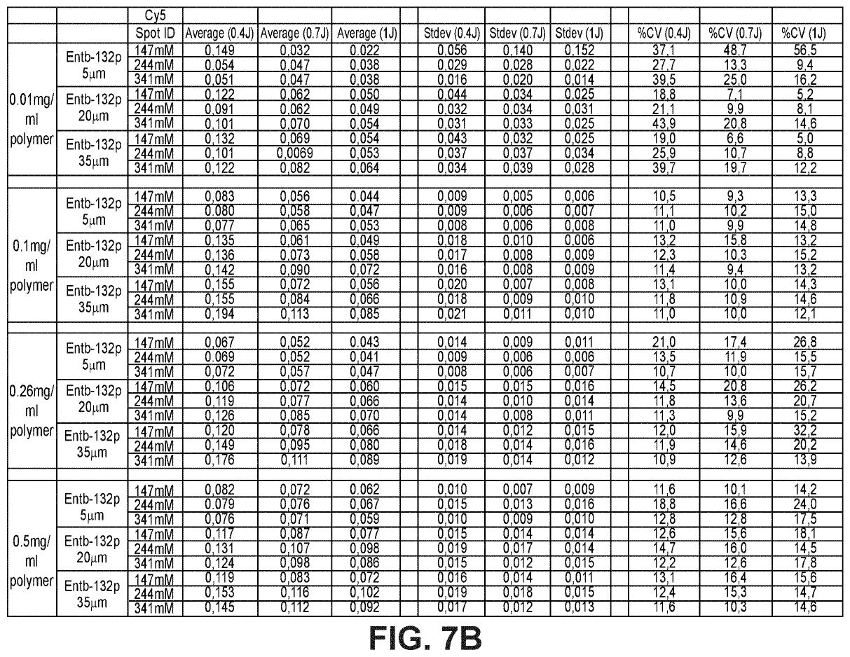

The polymer used to make a network of the disclosure can comprise at least one, at least two, or more than two cross-linker groups per molecule. A cross-linker group is a group that covalently bonds the polymer molecules of the network to each other and, optionally, to probes and/or a substrate. Copolymers that have been polymerized from two or more monomers (e.g., monomers having a polymerizable group independently selected from those described in the preceding paragraph), at least one of which comprises a cross-linker, are suitable for making a three-dimensional network of the disclosure. Exemplary cross-linkers are described in Section 5.1.2. A preferred monomer comprising a cross-linker is methacryloyloxybenzophenone (MABP) (see FIG. 7 of WO 2018/234253).

In a preferred embodiment, the copolymer is a bipolymer or a terpolymer comprising a cross-linker. In a particularly preferred embodiment, the copolymer comprises p(Dimethyacryamide co Methacryloyl-Benzophenone co Sodium 4-vinylbenzenesulfonate) (see FIG. 7 of WO 2018/234253). In some embodiments, the copolymer comprises dimethylacrylamide (DMAA), methacryloyloxybenzophenone (MABP), and sodium 4-vinylbenzenesulfonate (SSNa), where the polymer comprises 2.5 to 7.5 mol % MABP (e.g., 2.5 to 5 mol %, 3 to 6 mol % or 4 to 7.5 mol %), 2 to 5 mol % SSNa (e.g., 2 to 3 mol %, 2 to 4 mol %, or 3 to 5 mol %), and the balance DMAA. In some embodiments, the polymer comprises DMAA, MABP and SSNa in a 92.5:5:2.5 molar ratio.

Polymers of various molecular weights can be used in the processes for making three-dimensional networks described herein. For example, in some embodiments the average molecular weight of a polymer can range from 100 kDa to 600 kDa, e.g., 100 kDa to 500 kDa, 100 kDa to 400 kDa, 100 kDa to 300 kDa, 100 kDa to 200 kDa, 200 kDa to 600 kDa, 200 kDa to 500 kDa, 200 kDa to 400 kDa, 200 kDa to 300 kDa, 300 kDa to 600 kDa, 300 kDa to 500 kDa, 300 kDa to 400 kDa, 400 kDa to 600 kDa, 400 kDa to 500 kDa, or 500 kDa to 600 kDa. In some embodiments, the average molecule weight of the polymer is 300 kDa. Unless required otherwise by context, the term “average molecular weight” used herein refers to a weight average molecular weight.

In some embodiments, the concentration of polymer in the mixture (e.g., a copolymer of DMAA, MABP, and SSNa) is less than 1 mg/ml. For example, the concentration of the polymer can in some embodiments range from 0.01 mg/ml to less than 1 mg/ml, e.g., 0.01 mg/ml to 0.5 mg/ml, 0.01 mg/ml to 0.4 mg/ml, 0.01 mg/ml to 0.3 mg/ml, 0.01 mg/ml to 0.2 mg/ml, 0.01 mg/ml to 0.1 mg/ml, 0.05 mg/ml to 1 mg/ml, 0.05 mg/ml to 0.5 mg/ml, 0.05 mg/ml to 0.4 mg/ml, 0.05 mg/ml to 0.3 mg/ml, 0.05 mg/ml to 0.2 mg/ml, 0.05 mg/ml to 0.1 mg/ml, 0.1 mg/ml to 1 mg/ml, 0.1 mg/ml to 0.5 mg/ml, 0.1 mg/ml to 0.4 mg/ml, 0.1 mg/ml to 0.3 mg/ml, 0.1 mg/ml to 0.2 mg/ml. In some embodiments, the concentration of polymer is 0.1 mg/ml. Relatively low concentrations of polymer can reduce or eliminate premature precipitation of polymer during production of a three-dimensional polymer network.

### 5.1.2. Cross-Linkers

Cross-linking reagents (or cross-linkers) suitable for making the cross-links in the three-dimensional networks include those activated by ultraviolet light (e.g., short wave UV light or long wave UV light), visible light, and heat. Exemplary cross-linkers activated by UV light include benzophenone, thioxanthones (e.g., thioxanthen-9-one, 10-methylphenothiazine) and benzoin ethers (e.g., benzoin methyl ether, benzoin ethyl ether). Exemplary cross-linkers activated by visible light include ethyl eosin, eosin Y, rose bengal, camphorquinone and erythirosin. Exemplary cross-linkers activated by heat include 4,4′ azobis(4-cyanopentanoic) acid, and 2,2-azobis[2-(2-imidazolin-2-yl) propane]dihydrochloride, and benzoyl peroxide. Other cross-linkers known in the art, e.g., those which are capable of forming radicals or other reactive groups upon being irradiated, may also be used.

In preferred embodiments, cross-linker moieties comprise benzophenone moieties.

### 5.1.3. Salt

The polymer networks of the disclosure are characterized by transport channels that result when the polymers are cross-linked in a mixture containing salt crystals formed from an aqueous solution containing one or more types of salts, e.g., one, two, or at least two types of salts. In some embodiments, a salt forming needle shaped salt crystals (e.g., as described in WO 2017/103128) is used to make a three-dimensional network of the disclosure. In other embodiments, a salt forming needle shaped salt crystals and a salt forming cubic or rod shaped crystals (e.g., as described in WO 2018/234253) are used to make a three-dimensional network of the disclosure.

The one or more salts are preferably selected for their compatibility with one or more probes. Ideally, each salt has one or more of the following characteristics, (i) the salt is not toxic to the probes (e.g., the salt does not denature the probes), (ii) the salt does not react chemically with the probes, (iii) the salt does not attack fluorophores, such as cyanine dyes, which are suitable for the optical marking of probes, and/or (iv) the salt does not react with analytes, detection molecules, and/or binding partners bonded thereto. Preferably, at least one of the salts forms needle-shaped crystals.

In some embodiments, the salt solution comprises monovalent cations. The mixture can comprise disodium hydrogen phosphate and/or sodium dihydrogen phosphate which, in aqueous solution, releases Na⁺ cations and phosphate ions PO₄³⁻. Sodium phosphate is readily soluble in water and forms colorless crystals.

In some embodiments, the mixture comprises dipotassium hydrogen phosphate (K₂HPO₄) and/or potassium dihydrogen phosphate (KH₂PO₄). These salts are excellently soluble in water and can therefore form a correspondingly large number of needle-shaped salt crystals in the mixture.

In preferred embodiments, the aqueous salt solution comprises phosphate ions (e.g., a sodium phosphate solution) in a concentration ranging from 125 mM to less than 350 mM, e.g., 125 mM to 340 mM, 125 mM to 250 mM, 150 mM to 340 mM, 150 mM to 300 mM, 150 mM to 250 mM, 200 mM to 340 mM, 200 mM to 300 mM, 200 mM to 250 mM, 225 mM to 340 mM, 225 mM to 300 mM, or 225 mM to 250 mM. In some embodiments, the concentration of phosphate in the aqueous salt solution is 250 mM.

In some embodiments, the aqueous salt solution comprises a single type of monovalent cation, for example sodium or potassium cations.

In other embodiments, the aqueous salt solution comprises at least two types of monovalent cations, for example two types of alkali metal cations. Alkali metal cations that can be used include sodium cations and potassium cations, although other alkali metal cations, such as lithium cations, can also be used.

In some embodiments, the aqueous salt solution comprises sodium and potassium cations and/or has a total monovalent cation concentration such that when combined with the polymer solution and optional probe solution (prior to cross-linking) the resulting mixture has a total monovalent cation concentration of at least 500 mM. In particular embodiments, the sodium ion concentration in the mixture is at least 250 mM, and may range from 250 mM to 500 mM, more preferably is in the 300 mM to 400 mM range. In a specific embodiment, the sodium ion concentration in the mixture is 350 mM. In some embodiments, the potassium ion concentration in the mixture is preferably at least 150 mM, and preferably is in the range of 150 mM to 500 mM, more preferably is in the range of 200 mM to 400 mM, and yet more preferably is in the range of 250 mM to 350 mM.

In some embodiments, the aqueous salt solution can be a sodium phosphate buffer containing both disodium hydrogen phosphate and sodium dihydrogen phosphate, optionally supplemented with dipotassium hydrogen phosphate (K₂HPO₄) and/or potassium dihydrogen phosphate (KH₂PO₄). In one embodiment, a sodium phosphate buffer containing both disodium hydrogen phosphate and sodium dihydrogen phosphate and a potassium phosphate buffer containing both dipotassium hydrogen phosphate and potassium dihydrogen phosphate are made separately and combined into a single aqueous solution, prior to or after mixing with the polymer and/or probe solutions.

Generally, the aqueous salt solution preferably has a pH ranging from 6 to 9, and more preferably in the range of 7-8.5. In certain exemplary embodiments, the pH is 7.5, 8, or 8.5, most preferably 8.

For networks containing protein-based probe biomolecules, the aqueous salt solution can include phosphate buffered saline (“PBS”) and/or a monovalent cation sulfate.

### 5.1.4. Probes

Probes that can be immobilized on the network of the disclosure include biomolecules and molecule that binds biomolecules, e.g., a partner of a specifically interacting system of complementary binding partners (receptor/ligand). For example, probes can comprise nucleic acids and their derivatives (such as RNA, DNA, locked nucleic acids (LNA), and peptide nucleic acids (PNA)), proteins, peptides, polypeptides and their derivatives (such as glucosamine, antibodies, antibody fragments, and enzymes), lipids (e.g., phospholipids, fatty acids such as arachidonic acid, monoglycerides, diglycerides, and triglycerides), carbohydrates, enzyme inhibitors, enzyme substrates, antigens, and epitopes. Probes can also comprise larger and composite structures such as liposomes, membranes and membrane fragments, cells, cell lysates, cell fragments, spores, and microorganisms.

A specifically interacting system of complementary bonding partners can be based on, for example, the interaction of a nucleic acid with a complementary nucleic acid, the interaction of a PNA with a nucleic acid, or the enzyme/substrate, receptor/ligand, lectin/sugar, antibody/antigen, avidin/biotin or streptavidin/biotin interaction.

Nucleic acid probes can be a DNA or an RNA, for example, an oligonucleotide or an aptamer, an LNA, PNA, or a DNA comprising a methacyrl group at the 5′ end (5′ Acrydite™) Oligonucleotide probes can be, for example, 12 to 30, 14 to 30, 14 to 25, 14 to 20, 15 to 30, 15 to 25, 15 to 20, 16 to 30, 16 to 25, 16 to 20, 15 to 40, 15 to 45, 15 to 50, 15 to 60, 20 to 55, 18 to 60, 20 to 50, 30 to 90, 20 to 100, 20 to 60, 40 to 80, 40 to 100, 20 to 120, 20 to 40, 40 to 60, 60 to 80, 80 to 100, 100 to 120 or 12 to 150 nucleotides long. In preferred embodiments, the oligonucleotide probe is 15 to 60 nucleotides in length.

When using a nucleic acid probe, all or only a portion of the probe can be complementary to the target sequence. The portion of the probe complementary to the target sequence is preferably at least 12 nucleotides in length, and more preferably at least 15, at least 18 or at least 20 nucleotides in length. For nucleic acid probes of greater length than 40 or 50 nucleotides, the portion of the probe complementary to the target sequence can be at least 25, at least 30 or at least 35 nucleotides in length. When modified nucleic acid probes such as LNA or PNA are used, the portion of the probe complementary to the target sequence can in some embodiments be shorter than 12 nucleotides as these modified molecules have an increased binding energy to their complementary nucleic acid.

The antibody can be, for example, a polyclonal, monoclonal, or chimeric antibody or an antigen binding fragment thereof (i.e., “antigen-binding portion”) or single chain thereof, fusion proteins comprising an antibody, and any other modified configuration of the immunoglobulin molecule that comprises an antigen recognition site, including, for example without limitation, single chain (scFv) and domain antibodies (e.g., human, camelid, or shark domain antibodies), maxibodies, minibodies, intrabodies, diabodies, triabodies, tetrabodies, vNAR and bis-scFv (see e.g., Hollinger and Hudson, 2005, Nature Biotech 23:1126-1136). An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. “Antibody” also encompasses any of each of the foregoing antibody/immunoglobulin types.

Three-dimensional networks of the disclosure can comprise a single species of probe or more than one species of probe (e.g., 2, 3, 4, or 5 or more species). Three-dimensional networks can comprise more than one species of probe for the same target (e.g., antibodies binding different epitopes of the same target) and/or comprise probes that bind multiple targets.

The networks can comprise a labeled (e.g., fluorescently labeled) control probe molecule that can be used, for example, to measure the amount probe present in the network.

The probes can be distributed throughout the network (e.g., on a surface and the interior of a network). Preferably, at least one probe is spaced away from the surface of the network and adjoins at least one transport channel. A probe so located is then directly accessible for analyte molecules or analyte components through the transport channel. In some embodiments, a majority of the probes are located in the interior of the network.

The one or more probes can be immobilized on the network covalently or non-covalently. For example, a probe can be cross-linked to the cross-linked polymer or a probe can be non-covalently bound to the network (such as by binding to a molecule covalently bound to the network). In a preferred embodiment, one or more probes are cross-linked to the cross-linked polymer. In some embodiments, a majority of the probes are covalently bound in the interior of the network (e.g., such that at least a portion of the probes adjoin a transport channel).

Without being bound by theory, the inventors believe that the processes described herein for manufacturing three-dimensional networks in the presence of salt crystals (particularly phosphate salt crystals) may result in a greater concentration of probe molecule at or near the interface between the polymer and the transport channel due to electrostatic interactions between the probe molecules (particularly nucleic acid probe molecules) and the salt crystals. Accordingly, in some embodiments of the invention, the disclosure provides networks according to the disclosure in which the probe density is greater at the interface between the polymer and the transport channels than within regions of the polymer not abutting a transport channel. In various embodiments, the probe density it at least 10%, at least 20%, at least 30%, at least 40%, or at least 50% more dense at the interface between the polymer and the transport channels than within regions of the polymer not abutting a transport channel.

The density of probe molecule in a network can be verified using the following procedure:

The network is brought into contact with an aqueous liquid at room temperature, for example, in a bowl. The liquid contains a plurality of nanoparticles attached to a moiety that interacts with the probe molecules in the network, for example streptavidin if the probe molecules are biotinylated. The size of the nanoparticles is smaller than the mesh size of the network and smaller than the minimum cross-section of at least one type of transport channel in the network to allow the nanoparticles to become distributed throughout the polymer. Suitable nanoparticles are quantum dots 2-5 nanometers in dimeter.

An incubation period is selected so that the network in the liquid is completely hydrated, i.e., that the network on average takes the same amount of water as it releases. The incubation period can be, for example, one hour. The penetration of the nanoparticles in the network can be accelerated by setting in motion the network and/or the liquid during the incubation, for example, by vibrating the network and/or liquid, preferably by means of ultrasonic waves.

After completion of the incubation, the liquid is separated from the network, for example, by draining the liquid from the bowl or taking the network out of the bowl.

Then, the hydrated network is frozen, for example, by means of liquid nitrogen. Thereafter, the frozen network can be cut with the aid of a cryomicrotome along mutually parallel cutting planes into thin slices. The cutting planes are arranged transversely to the longitudinal extension of the transport channel and penetrate the transport channel. The cutting is preferably carried out using a liquid nitrogen-cooled diamond blade. The thickness of the slices can be, for example, about 100 nm or 200 nm.

With the aid of a microscope, the nanoparticles disposed in the disks obtained by cutting the frozen network are located. The nanoparticles can be fluorescent and optically highlighted so that they can be better distinguished from the network, if necessary. The locating of the nanoparticles can be done using a suitable software with image processing methods. To examine the disks, preferably a confocal microscope laser scanning microscope with fluorescence optics or an electron microscope is used.

The geometry and/or position information of the nanoparticles obtained in this manner may be, with the aid of a computer, used to make a three-dimensional geometric model of distribution of the nanoparticles in the network. The model can then be used to determine whether the distribution of nanoparticles reflects a greater density of probe molecules near sites of transport channels.

When the mixture used in the production of a three-dimensional network includes oligonucleotide probe molecules, the concentration of the probe molecules in some embodiments ranges from 5 μM to 35 μM, e.g., 15 μM to 25 μM, 5 μM, 20 μM, or 35 μM, or any range bounded by any two of the foregoing values.

### 5.1.5. Salt Crystal Forming Conditions

Salt crystal forming conditions can comprise dehydrating the mixture or cooling the mixture until the relative salt content in the mixture increases to above the solubility limit, meaning that the mixture is supersaturated with the salt. This promotes the formation of salt crystals from a crystallization germ located in the volume of the mixture towards the surface of the mixture. It is believed, without being bound by theory, that the use of aqueous solutions containing at least two different monovalent metal ions results in the formation of at least two different types of salt crystals.

The mixture can be dehydrated by heating the mixture, exposing the mixture to a vacuum, and/or reducing the humidity of the atmosphere surrounding the mixture.

The mixture can be heated by placing the mixture on a heated substrate or surface (e.g., between about 50° C. to about 70° C.), heating the substrate or surface on which the mixture has been placed (e.g., to between about 50° C. to about 70° C.), and/or contacting the mixture with a hot gas (e.g., air, nitrogen, or carbon dioxide having a temperature that is higher than the temperature of the mixture) such that water is evaporated from the mixture. The contacting with the hot gas can, for example, take place by placing the mixture in a heating oven. During the transportation to the heating oven, the mixture can be kept at a humidity of 40% or greater, for example at a relative humidity of approximately 60%, although higher relative humidities, even as high as 75% or greater, are also feasible. Mixtures with higher potassium ion concentrations can tolerate lower relative humidities, and mixtures with lower potassium salt concentration are preferably kept at higher relative humidities during transport.

By heating the mixture it is also possible to activate thermally activatable cross-linkers, if present, and cross-link the polymer thereby.

In some embodiments, the temperature of the heated substrate and/or air used to dehydrate the mixture is 20° C. or more above the temperature of the mixture before heating the mixture, but less than 100° C.

The mixture can be cooled by placing the mixture on a cooled substrate or surface (e.g., between about 5° C. to about 15° C.), cooling the substrate or surface on which the mixture has been placed (e.g., to between about 5° C. to about 15° C.) and/or bringing it into contact with a cold gas (e.g., air, nitrogen, or carbon dioxide having a temperature that is lower than the temperature of the mixture). When cooled, the temperature-dependent solubility limit of the salt in the mixture decreases until the mixture is ultimately supersaturated with the salt. In some embodiments, the mixture is cooled by incubating it in a cold chamber with low humidity (e.g., temperatures between 0° C. and 10° C., relative humidity <40%).

The temperature in the mixture is preferably held above the dew point of the ambient air surrounding the mixture during the formation of the one or more salt crystals. This prevents the mixture becoming diluted with water condensed from the ambient air, which could lead to a decrease in the relative salt content in the mixture.

### 5.1.6. Cross-Linking Conditions

The cross-linking conditions can be selected based upon the type of cross-linker used. For example, when using a cross-linker activated by ultraviolet light (e.g., benzophenone, a thioxanthone or a benzoin ether), the cross-linking conditions can comprise exposing the mixture to ultraviolet (UV) light. In some embodiments, UV light having a wavelength from about 250 nm to about 360 nm is used (e.g., 260±20 nm or 355±20 nm). The use of lower energy/longer wavelength UV light (e.g., 360 nm UV light vs. 254 nm UV light) can require longer exposure times. When using a cross-linker activated by visible light (e.g., ethyl eosin, eosin Y, rose bengal, camphorquinone or erythirosin), the cross-linking conditions can comprise exposing the mixture to visible light. When using a thermally activated cross-linker (e.g., 4,4′ azobis(4-cyanopentanoic) acid, and 2,2-azobis[2-(2-imidazolin-2-yl) propane]dihydrochloride, or benzoyl peroxide), the cross-linking conditions can comprise exposing the mixture to heat.

The length and intensity of the cross-linking conditions can be selected to effect cross-linking of polymer molecules to other polymer molecules, cross-linking of polymer molecules to probe molecules (if present), and cross-linking of polymer molecules to substrate molecules or organic molecules present on the substrate (if present). The length and intensity of cross-linking conditions for a mixture containing probes can be determined experimentally to balance robustness of immobilization and nativity of probe molecules, for example.

In some embodiments, when UV cross-linking is used (e.g., cross-linking with a 254 nm UV light), the dose of cross-linking energy is less than 1 J/cm². For example, in some embodiments, the cross-linking energy is 0.4 J/cm² to 0.9 J/cm², 0.5 J/cm² to 0.9 J/cm², 0.6 J/cm² to 0.9 J/cm², 0.7 J/cm² to 0.9 J/cm², 0.4 J/cm² to 0.8 J/cm², 0.5 J/cm² to 0.8 J/cm², 0.6 J/cm² to 0.8 J/cm², 0.7 J/cm² to 0.8 J/cm², 0.4 J/cm² to 0.7 J/cm², 0.5 J/cm² to 0.7 J/cm², 0.6 J/cm² to 0.7 J/cm², 0.4 J/cm² to 0.6 J/cm², 0.5 J/cm² to 0.6 J/cm², or 0.4 J/cm² to 0.5 J/cm². In some embodiments, a UV energy dose of 0.7 J/cm² is used.

### 5.1.7. Salt Crystal Dissolution

After cross-linking the polymer, the salt crystals can be dissolved in the solvent in such a way that at least one transport channel is formed in the network.

When the crystals include needle-shaped salt crystals, elongated channels extending from the surface and/or near the surface of the network into the interior of the network can be formed. It is believed, without being bound by theory, that the use of two types of monovalent salt cations during crystal formation results in at least two types of crystals, compact crystals and needle-shaped crystals. The dissolution of the compact crystals is believed to result in short channels that create a sponge-like effect in the network, pierced by long channels resulting from the dissolution of the needle-shaped crystals.

When using an array produced by the method of the disclosure as a biological sensor, a high measurement accuracy and high measurement dynamic are permitted.

The solvent for dissolving the one or more salt crystals can be chosen in such a way that it is compatible to the polymer and probes, if present (e.g., the solvent can be chosen such that it does not dissolve the polymer and probes). Preferably, the solvent used is a water based buffer, such as diluted phosphate buffer. Methanol, ethanol, propanol or a mixture of these liquids can be added to the buffer to facilitate the removal of unbound polymer from the network.

After the removal of the salt crystals the network can collapse due to drying and can be rehydrated. Drying the network has advantages for shipping and stabilization of probe biomolecules.

### 5.2. Three-Dimensional Polymer Networks and Pluralities of Three-Dimensional Polymer Networks

In one aspect, the disclosure provides three-dimensional polymer networks made by a process described herein. The three-dimensional networks comprise a cross-linked polymer, one or more transport channels and can optionally further comprise one or more probes immobilized on the network, e.g., by cross-linking to the polymer chains. Probes that can be immobilized on the networks are described in Section 5.1.4.

The networks of the disclosure can have a mesh size (measured in the hydrated state of the network) of, for example, 5 to 75 nm (e.g., 10 to 20 nm, 10 to 30 nm, 10 to 40 nm, 10 to 50 nm, 20 to 30 nm, 20 to 40 nm, 20 to 50 nm, 30 to 40 nm, 30 to 50 nm, or 40 to 50 nm). The “hydrated state of the network” means that the network is at equilibrium with respect to water absorption, i.e., it absorbs in aqueous solution as much water as it emits.

Transport channels can allow access to the interior of the network. Although transport channels can have a relatively large cross-section, the network can remain mechanically stable because the mesh size of the network can be significantly smaller than the transport channel cross-section.

The transport channels can form a sort of highway, through which analytes can enter quickly in and out of the interior of the network. The transport of the analytes can be effected in the transport channels by diffusion and/or convection.

Transport channels are formed when a network is formed by cross-linking polymer chains in the presence of salt crystals, as described in Section 5.1. After salt crystals are washed away, transport channels are left behind.

Three dimensional networks of the disclosure can include one or more types of transport channels. When the salt crystals formed in the processes for making three-dimensional hydrogel networks described herein are washed away, transport channels are left behind, according to the principle of the “lost” form. The transport channels allow analytes to penetrate into the interior of the network and specifically bind a probe located in the interior of the network. Additionally, the transport channels allow unbound analytes to exist the interior of the network after washing, reducing the amount of nonspecific signal from analytes “stuck” within the network.

One type of transport channel that can be present in a three-dimensional network of the disclosure is believed to be a long channel created from needle-shaped salt crystals. As used herein, a “long channel” is an elongated passage in a network that (1) is substantially straight, and (2) in the hydrated state of the network, has a minimum cross-section that is at least 300 nm and a length that is at least three times, preferably five times, and more preferably at least ten times, the minimum cross-section of the passage. For example, the length of the long channel can be 3 to 15 times, 5 to 10 times, or 10 to 15 times the minimum cross-section of the long channel. A long channel that is “substantially straight” is one which extends from a point of nucleation in one direction without changing direction more than 45 degrees in any direction, i.e., the X, Y or Z direction. Because long channels arise from needle-shaped crystals that form from a common nucleation point, the networks of the disclosure might include groups of (e.g., 5, 10 or more) long channels that converge at a point located within the network corresponding to the original nucleation point of crystallization. Long channels are typically arranged such that, starting from the surface of the network towards the interior, the lateral distance between the long channels decreases.

Another type of transport channel that can be present in a three-dimensional network of the disclosure is believed to be a short channel, for example formed from cubic or rod-shaped crystals. As used herein, a “short channel” is a passage in a network that (1) is substantially straight, and (2) in the hydrated state of the network, has a minimum cross-section that is preferably at least 10 times the mesh size of the network and a length that is less than three times (e.g., can range from 1 time to 2.75 times, from 1 time to 2.5 times, from 1 time to 2 times, or from 1 time to 1.5 times) the minimum cross-section of the passage. A short channel that is “substantially straight” is one which extends from a point of nucleation in one direction without changing direction more than 45 degrees in any direction, i.e., the X, Y or Z direction. To maintain network strength, a short channel preferably has a cross-section of no greater than 1/20^{th} of the network width or diameter, for example for a network that is in the form of a “spot” on an array with a diameter of 200 μm, the cross-section of the short channel is preferably no greater than 10 μm, and for a spot on an array with a diameter of 100 μm, the cross-section of the short channel is preferably no greater than 5 μm. In certain aspects, the cross-section of the short channel is about 20 nm or greater, about 50 nm or greater, about 100 nm or greater, about 250 nm or greater, at least 500 nm or greater, or about 1 μm or greater. The short channels in a network can have approximately (e.g., +/−10% or +/−25%) the same diameter or different diameters. In particular embodiments, the short channels in a network have a diameter ranging between any two of the foregoing dimensions, e.g., they can range from 100 nm to 10 μm, from 50 nm to 1 μm, from 500 nm to 5 μm, from 250 nm to 10 μm, and so on and so forth.

Without being bound by theory, the inventors believe that a sponge polymer having short channels penetrated by long channels can be created when the mixture used to make the network includes an aqueous salt solution having components that can form different metal ion-salt ion pairings.

In some embodiments, a three-dimensional network of the disclosure includes long and short channels. In other embodiments, a three-dimensional network of the disclosure includes only long channels (e.g., when a single species of salt is included in the aqueous salt solution).

In another aspect, the disclosure provides pluralities of three-dimensional polymer networks described herein (e.g., pluralities of two or more, five or more, 10 or more, or 20 or more and/or up to 50, up to 100, or up to 1000). In some embodiments, the individual members of a plurality of three-dimensional polymer networks are positioned on a single array; in other embodiments, the individual members are positions one two or more separate arrays. For example, an array can be an array as described in Section 5.3.

The members of a plurality of three-dimensional networks can have a high degree of uniformity with each other. For example, when contacted with an analyte (e.g., a fluorescently labeled oligonucleotide) capable of binding to a probe molecule present in the three-dimensional networks of a plurality, the measured signals for the three-dimensional networks can be relatively similar, e.g., having a coefficient of variation of less than 25%, 20%, 15%, 10%, 9%, 8%, 7%, 6%, or 5%. In some embodiments, the coefficient of variation is less than 25% but at least 1%, at least 2%, at least 5%, or at least 10%; or less than 20% but at least 1%, at least 2%, at least 5%, or at least 10%; or less than 15% but at least 1%, at least 2%, at least 5%, or at least 10%; or less than 10% but at least 1%, at least 2%, or at least 5%; or less than 9% but at least 1%, at least 2%, or at least 5%; less 8% but at least 1%, at least 2%, or at least 5%; less than 7% but at least 1%, at least 2%, or at least 5%; less than 6% but at least 1%, at least 2%, or at least 5%; or less 5% but at least 1% or at least 2%.

For pluralities of networks having fluorescently labeled probes, the probes can be used to assess uniformity without (or in addition to) binding a labeled analyte. In some embodiments, when exciting a fluorescently labeled probe present in the three-dimensional networks of a plurality, the coefficient of variation for measured signals is less than 25%, 20%, 15%, 10%, 9%, 8%, 7%, 6%, or 5%. In some embodiments, the coefficient of variation is less than 25% but at least 1%, at least 2%, at least 5%, or at least 10%; or less than 20% but at least 1%, at least 2%, at least 5%, or at least 10%; or less than 15% but at least 1%, at least 2%, at least 5%, or at least 10%; or less than 10% but at least 1%, at least 2%, or at least 5%; or less than 9% but at least 1%, at least 2%, or at least 5%; less 8% but at least 1%, at least 2%, or at least 5%; less than 7% but at least 1%, at least 2%, or at least 5%; less than 6% but at least 1%, at least 2%, or at least 5%; or less 5% but at least 1% or at least 2%.

### 5.3. Arrays

The three-dimensional networks of the disclosure can be positioned (e.g., deposited) on a substrate, and are preferably immobilized on a substrate (e.g., by covalent cross-links between the network and the substrate). A plurality of networks can be immobilized on a substrate to form an array useful, for example, as a biochip.

Suitable substrates include organic polymers, e.g., cycloolefin copolymers (COCs), polystyrene, polyethylene, polypropylene, polycarbonate, and polymethylmethacrylate (PMMA, Plexiglas®). Ticona markets an example of a suitable COC under the trade name Topas®. Inorganic materiels (e.g., metal, glass) can also be used as a substrate. Such substrates can be coated with organic molecules to allow for cross-links between the network and a surface of the substrate. For example, inorganic surfaces can be coated with self-assembled monolayers (SAMs). SAMs can themselves be completely unreactive and thus comprise or consist of, for example, pure alkyl silanes. Other substrates can also be suitable for cross-linking to the three-dimensional network provided they are able to enter into stable bonds with organic molecules during free-radical processes (e.g., organoboron compounds).

The substrate can be rigid or flexible. In some embodiments, the substrate is in the shape of a plate (e.g., a rectangular plate, a square plate, a circular disk, etc.). For example, the substrate can comprise a microwell plate, and the three-dimensional networks can be positioned in the wells of the plate.

The individual networks can be positioned at distinct spots on a surface of the substrate, e.g., in a matrix comprising a plurality of columns and rows. Arrays having different numbers of rows and columns, the number of each of which can be independently selected, are contemplated (e.g., 2 to 64 columns and 2 to 64 rows). The columns can be separated by a distance X and the rows can be separated by a distance Y (for example, as shown in FIG. 9 of WO 2018/234253) so as to form a grid of spots on which the individual networks can be located. X and Y can be selected so that the networks, located at the spots of the grid, do not contact each other in the dehydrated state and do not contact each other in the hydrated state. The dimensions X and Y can be the same or different. In some embodiments, X and Y are the same. In some embodiments, X and Y are different. In some embodiments, X and Y are independently selected from distances of at least about 500 μm (e.g., 500 μm to 5 mm, 500 μm to 4 mm, 500 μm to 3 mm, 500 μm to 2 mm, or 500 μm to 1 mm). In some embodiments, X and Y are both about 500 μm. In other embodiments, X and Y are both 500 μm.

In some embodiments, substrate is band-shaped (for example, as shown in FIG. 10 of WO 2018/234253). The networks can be arranged as a single row extending in the longitudinal direction of a band-shaped organic surface, or can be arranged as multiple rows extending in the longitudinal direction of the band-shaped surface. The rows and columns in such band-shaped arrays can have grid dimensions X and Y as described above.

The individual networks can each cover an area of the surface of the array that is circular or substantially circular. Typically, the diameter of the area on the surface of the array covered by the individual networks (i.e., the spot diameter) is 80 μm to 1000 μm. In various embodiments, the spot diameter is 80 μm, 100 μm, 120 μm, 140 μm, 160 μm, 180 μm, 200 μm, 300 μm, 400 μm, 500 μm, 600 μm, 700 μm, 800 μm, 900 μm, or 1000 μm, or selected from a range bounded by any two of the foregoing embodiments, e.g., 80 μm to 200 μm, 100 μm to 120 μm, 120 μm to 140 μm, 120 μm to 180 μm, 140 μm to 160 μm, 160 μm to 180 μm, 180 μm to 200 μm, 120 μm to 200 μm, 100 μm to 400 μm, 160 μm to 600 μm, or 120 μm to 700 μm, and so on and so forth. In a preferred embodiment, the diameter ranges from 100 μm to 200 μm or a subrange thereof.

The arrays of the disclosure typically have at least 8 individual three-dimensional networks. In certain aspects, the arrays have at least 16, at least 24, at least 48, at least 96, at least 128, at least 256, at least 512, or at least 1024 individual three-dimensional networks. In some embodiments, the arrays of the disclosure have 24, 48, 96, 128, 256, 512, 1024, 2048, 4096 or 8192 individual networks, or have a number of three-dimensional networks selected from a range bounded any two of the foregoing embodiments, e.g., from 8 to 128, 8 to 512, 24 to 8192, 24 to 4096, 48 to 2048, 96 to 512, 128 to 1024, 24 to 1024, 48 to 512, 96 to 1024, or 128 to 512 three-dimensional networks, and so on and so forth. In a preferred embodiment, number of three-dimensional networks on an array ranges from 8 to 1024. In a particularly preferred embodiment, the number of three-dimensional networks on an array ranges from 25 to 400.

The individual networks which comprise the arrays of the disclosure can have identical or different probes (e.g., each network can have a unique set of probes, multiple networks can have the same set of probes and other networks can have a different set or sets of probes, or all of networks can have the same set of probes). For example, networks arranged in the same row of a matrix can comprise the same probes and the networks arranged in different rows of the matrix can have different probes.

Typically, the individual networks on an array vary by no more than 20%, no more than 15%, no more than 10% or no more than 5% from one another by spot diameter and/or network volume.

In some embodiments, the arrays comprise one or more individual networks (e.g., spots on an array) with one or more control oligonucleotides or probe molecules. The control oligonucleotides can be labelled, e.g., fluorescently labelled, for use as a spatial control (for spatially orienting the array) and/or a quantifying the amount of probe molecules bound to the networks, for example, when washing and reusing an array of the disclosure (i.e., as a “reusability control”). The spatial and reusability control probes can be the same or different probes.

The same spot on the array or a different spot on the array can further include an unlabelled probe that is complementary to a known target. When used in a hybridization assay, determining the signal strength of hybridization of the unlabelled probe to the labelled target can determine the efficiency of the hybridization reaction. When an individual network (i.e., a spot on an array) is used both as a reusability and/or spatial control and a hybridization control, a different fluorescent moiety can be used to label the target molecule than the fluorescent moiety of the reusability control or spatial control probes.

In some embodiments, the arrays of the disclosure can be reused at least 5 times, at least 10 times, at least 20 times, at least 30 times, at least 40 times, or at least 50 times (e.g., 5 to 20 times, 5 to 30 times, 10 to 50 times, 10 to 20 times, 10 to 30 times, 20 to 40 times, or 40 to 50 times, preferably comprising reusing the array 10 to 50 times). The array can be washed with a salt solution under denaturating conditions (e.g., low salt concentration and high temperature). For example, the array can be washed with a 1-10 mM phosphate buffer at 80-90° C. between uses. The temperature of the wash can be selected based upon the length (Tm) of the target:probe hybrid.

The integrity of an array can be determined by a “reusability control” probe. The reusability control probe can be fluorescently labeled or can be detected by hybridization to a fluorescently labeled complementary nucleic acid. The fluorescent label of a fluorescently labeled reusability control probe may be bleached by repeated excitation, before the integrity of the nucleic acid is compromised; in such cases any further reuses can include detection of hybridization to a fluorescently labeled complementary nucleic acid as a control. Typically, an array of the invention is stable for at least 6 months.

In various embodiments, a fluorescently labeled reusability control probe retains at least 99%, 95% 90%, 80%, 70%, 60%, or 50% of its initial fluorescence signal strength after 5, 10, 20, 30, 40, or 50 uses. Preferably, the reusability control probe retains least 75% of its fluorescence signal strength after 5 or 10 uses. An array can continue to be reused until the reusability control probe retains at least 50% of its fluorescence signal strength, for example after 20, 30, 40 or 50 reuses. The fluorescent signal strength of the control probe can be tested between every reuse, every other reuse, every third reuse, every fourth reuse, every fifth reuse, every sixth reuse, every seventh reuse, every eighth reuse, every ninth reuse, every tenth reuse, or a combination of the above. For example, the signal strength can be tested periodically between 5 or 10 reuses initially and the frequency of testing increased with the number of reuses such that it is tested after each reuse after a certain number (e.g., 5, 10, 20, 30, 40 or 50) uses. In some embodiments, the frequency of testing averages once per 1, 1.5, 2, 2.5, 3, 4, 5 or 10 uses, or averages within a range bounded between any two of the foregoing values, e.g., once per 1-2 uses, once per 1-1.5 uses, once per 1-3 uses, or once per 1.5-3 uses.

It is noted that the nomenclature of “spatial control”, “reusability control” and “hybridization control” is included for convenience and reference purposes and is not intended to connote a requirement that the probes referred to “spatial control”, “reusability control” and “hybridization control” be used as such.

### 5.4. Methods of Using the Three-Dimensional Networks

The networks and arrays of the disclosure can be used to determine the presence or absence of an analyte in a sample, preferably a liquid sample. The disclosure therefore provides methods for determining whether an analyte is present in a sample or plurality of samples, comprising contacting a network or array of the disclosure comprising probe molecules that are capable of binding to the analyte with the sample or plurality of samples and detecting binding of the analyte to the probe molecules, thereby determining whether the analyte is present in the sample or plurality of samples. When arrays comprising different species of probes capable of binding different species of analyte are used in the methods, the presence of the different species of analytes can be determined by detecting the binding of the different species of analytes to the probes. In some embodiments, the methods further comprise a step of quantifying the amount of analyte or analytes bound to the array.

The analyte can be, for example, a nucleic acid, such as a polymerase chain reaction (PCR) amplicon. In some embodiments, the PCR amplicon is amplified from a biological or environmental sample (e.g., blood, serum, plasma, tissue, cells, saliva, sputum, urine, cerebrospinal fluid, pleural fluid, milk, tears, stool, sweat, semen, whole cells, cell constituent, cell smear, or an extract or derivative thereof). In some embodiments, the nucleic acid is labeled (e.g., fluorescently labeled).

An analyte placed on the surface of the network can penetrate into the interior of the network through the transport channel in order to specifically bind to a probe (e.g., a biomolecule) covalently bonded there to the polymer. When using the arrays of the disclosure with the networks immobilized thereon as biological sensor, a high measurement accuracy and also a high measurement dynamic is permitted.

The networks and arrays of the disclosure can be regenerated after use as a biosensor and can be used several times (e.g., at 5 times, at least 10 times, at least 20 times, at least 30 times, at least 40 times, or at least 50 times). If the probe molecules are DNA, this can be achieved, for example, by heating the network(s) in an 1× phosphate buffered saline to a temperature between 80° C. and 90° C. for about 10 minutes. Then, the phosphate buffered saline can be exchanged for a new phosphate buffered saline to wash the denatured DNA out of the network(s). If the probe molecules of the network(s) or array are antigens the network(s) or array can be regenerated by bringing the network(s) into contact with 0.1 N NaOH for about 10 minutes. Then, the 0.1 N NaOH can be exchanged for a phosphate buffered saline to wash the antigens out of the network. Thus, some embodiments of the methods of using the networks and arrays of the disclosure comprise using a network or array that has been washed prior to contact with a sample or a plurality of samples.

### 5.5. Applications of Arrays of the Disclosure

Because the arrays of the invention achieve economical determination of the qualitative and quantitative presence of nucleic acids in a sample, it has immediate application to problems relating to health and disease in human and non-human animals.

In these applications a preparation containing a target molecule is derived or extracted from biological or environmental sources according to protocols known in the art. The target molecules can be derived or extracted from cells and tissues of all taxonomic classes, including viruses, bacteria and eukaryotes, prokaryotes, protista, plants, fungi, and animals of all phyla and classes. The animals can be vertebrates, mammals, primates, and especially humans. Blood, serum, plasma, tissue, cells, saliva, sputum, urine, cerebrospinal fluid, pleural fluid, milk, tears, stool, sweat, semen, whole cells, cell constituent, and cell smears are suitable sources of target molecules.

The target molecules are preferably nucleic acids amplified (e.g., by PCR) from any of the foregoing sources).

The arrays of the invention can include probes that are useful to detect pathogens of humans or non-human animals. Such probes include oligonucleotides complementary at least in part to bacterial, viral or fungal targets, or any combinations of bacterial, viral and fungal targets.

The arrays of the invention can include probes useful to detect gene expression in human or non-human animal cells, e.g., gene expression associated with a disease or disorder such as cancer, cardiovascular disease, or metabolic disease for the purpose of diagnosing a subject, monitoring treatment of a subject or prognosis of a subject's outcome. Gene expression information can then track disease progression or regression, and such information can assist in monitoring the success or changing the course of an initial therapy.

### 5.6. Kits

In another aspect, the disclosure provides kits comprising a mixture comprising an aqueous salt solution as described herein, a polymer as described herein, cross-linker moieties as described herein (which can be covalently attached to the polymer), a substrate as described herein, and, optionally probe molecules as described herein. The kits of the disclosure can be used, for example, to make a three-dimensional network and/or array as described herein.

### 6. EXAMPLES

### 6.1. Example 1: Effect of Process Parameters on Three-Dimensional Networks and Arrays: Study 1

When making arrays having three-dimensional networks with transport channels according to the processes described in WO 2017/103128 and using a mixture comprising sodium phosphate with a phosphate concentration of 341 mM, 1 mg/ml of the cross-linking polymer poly(dimethylacrylamide) co 5% Methacryloyl-Benzophenone co 2.5% Sodium 4-vinylbenzenesulfonate, and oligonucleotide probes, variability among the resulting three-dimensional networks was observed. When investigating the cause of the variability, it was surprisingly discovered that polymer was sometimes prematurely precipitating from the mixture before formation of salt crystals, forming a cap of polymer on top of the mixture. It was further discovered that the polymer caps would sometimes, but not always, be removed during subsequent washing, contributing to variability among three-dimensional networks on a given array.

Following the discovery that polymer could prematurely separate from the mixture, the inventors investigated the effect of polymer concentration, buffer (salt) concentration, cross-linking energy, and oligonucleotide probe concentration on three-dimensional network and array variability. Specifically, the following parameters were evaluated:

TABLE 1

| Factor | Values evaluated |
|---|---|
| Polymer concentration | 0.01 mg/ml, 0.1 mg/ml, |
| (poly(dimethylacrylamide) co 5% | 0.26 ml/ml, 0.5 mg/ml |
| Methacryloyl-Benzophenone co 2.5% | |
| Sodium 4-vinylbenzenesulfonate) | |
| Phosphate concentration | 147 mM, 244 mM, and 341 mM |
| (sodium phosphate) | |
| Oligonucleotide probe concentration | 5 μM, 20 μM, and 35 μM |
| (probes AllCan1 and Sau-71p) | |
| Cross-linking energy | 0.4 J/cm², 0.7 J/cm², 1 J/cm² |

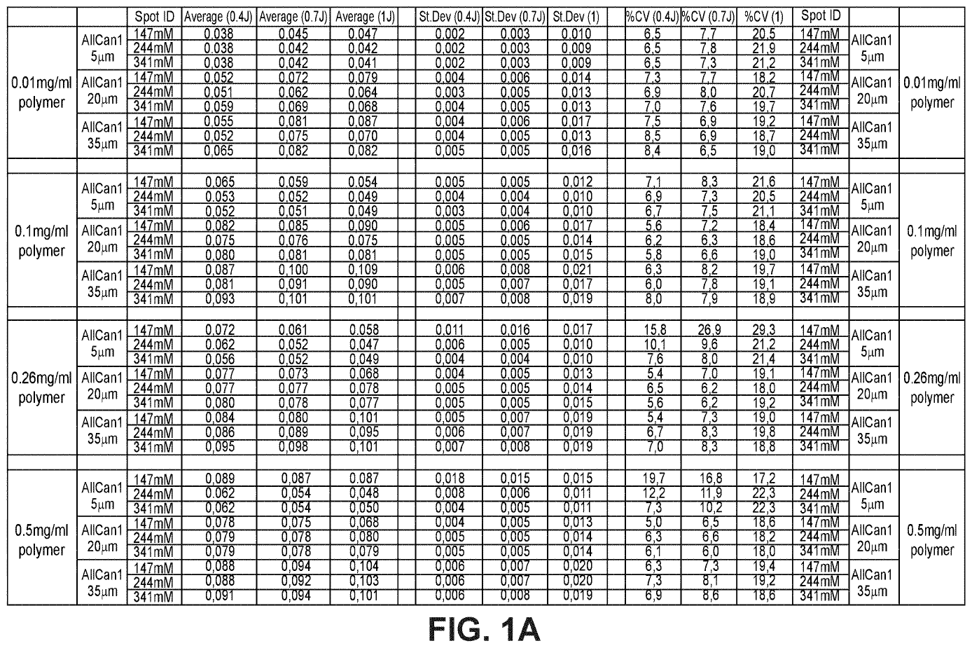

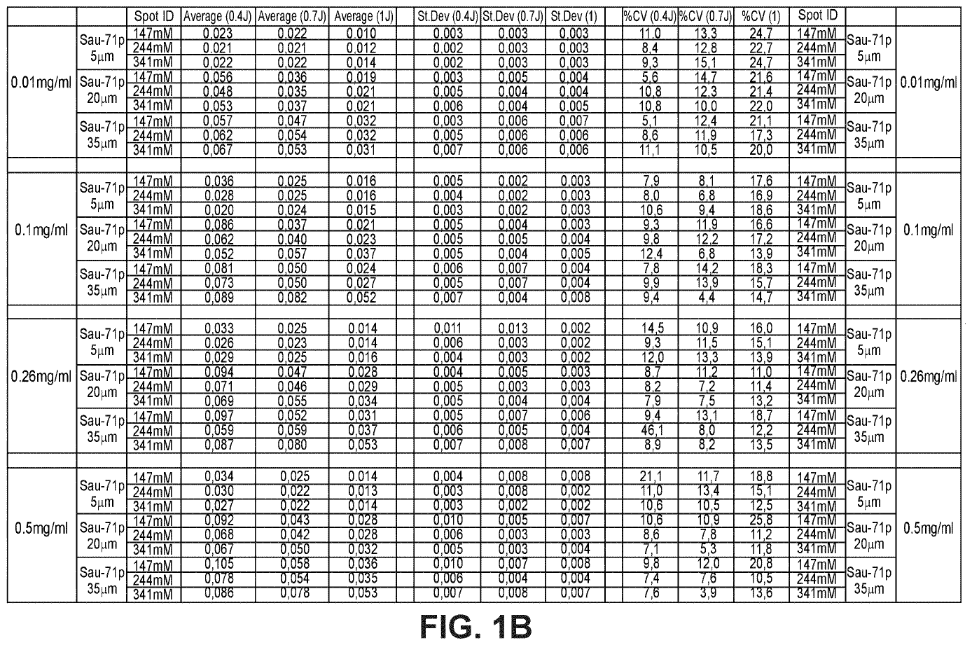

Signal intensities and coefficients of variation (CV) were measured for three-dimensional networks made with the different polymer concentrations, phosphate concentrations, probe concentrations, and cross-linking energies. Results are shown in FIG. 1A-1B.

It was discovered that the most significant factors for the variability observed using the original mixture was the polymer concentration and cross-linking energy. A high polymer concentration was found to result in relatively high amounts of three-dimensional networks that were not homogeneous. Without being bound by theory, this was attributed to the formation of a polymer precipitate in the mixture and to incomplete extraction of the precipitate after cross-linking. Again without being bound by theory, it is believed that the use of lower crosslinking energies decrease the extent of cross-linking, which facilitates the extraction of unbound material, thus making the three-dimensional networks on the array more homogeneous.

It was further discovered that lowering the phosphate concentration also improved variation among three-dimensional networks (lowered CV) and reduced the risk of premature precipitation of components from the mixture.

### 6.2. Example 2: Effect of Process Parameters on Three-Dimensional Networks and Arrays: Study 2

A study similar to Example 1 was performed using probes E. coli-1637p and Entb-132p.

TABLE 2

| Factor | Values evaluated |
|---|---|
| Polymer concentration | 0.01 mg/ml, 0.1 mg/ml, |
| (poly(dimethylacrylamide) co 5% | 0.26 ml/ml, 0.5 mg/ml |
| Methacryloyl-Benzophenone co 2.5% | |
| Sodium 4-vinylbenzenesulfonate) | |
| Phosphate concentration | 147 mM, 244 mM, and 341 mM |
| (sodium phosphate) | |
| Oligonucleotide probe concentration | 5 μM, 20 μM, and 35 μM |
| (Cy3 labeled probes E.coli-1637p and | |
| Entb-132p) | |
| Cross-linking energy | 0.4 J/cm² (columns 1, 4, 7, 10); |
| | 0.7 J/cm² (columns 2, 5, 8, 11); |
| | 1 J/cm² (columns 3, 6, 9, and 12) |

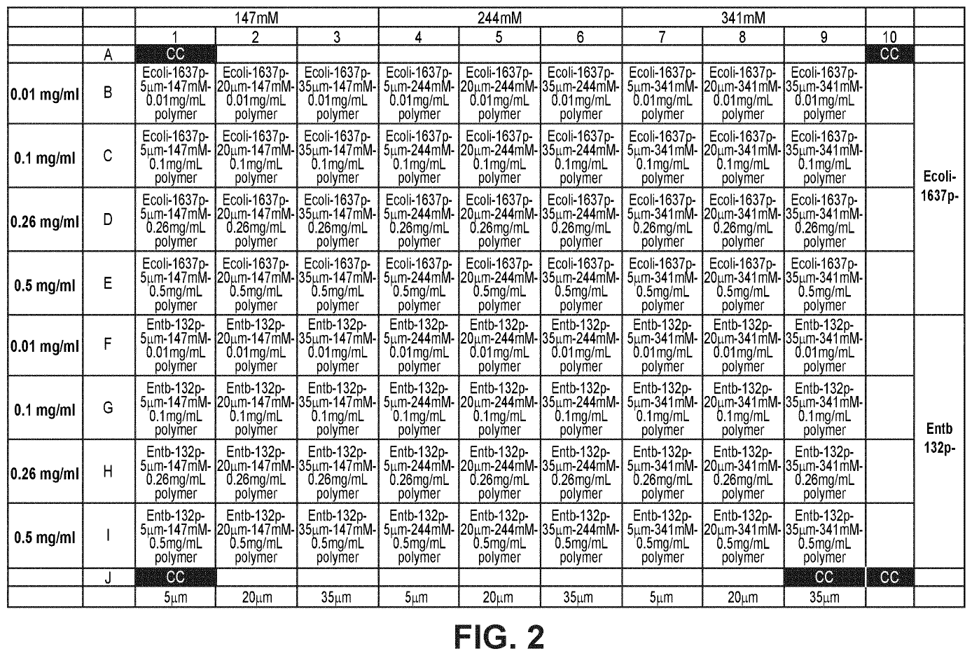

The print plan for making the three-dimensional networks of the study is shown in FIG. 2A.

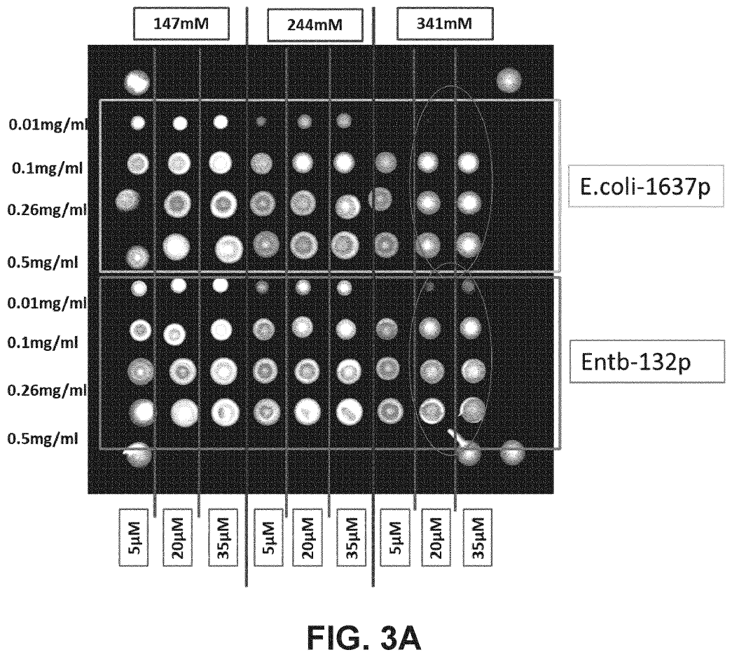

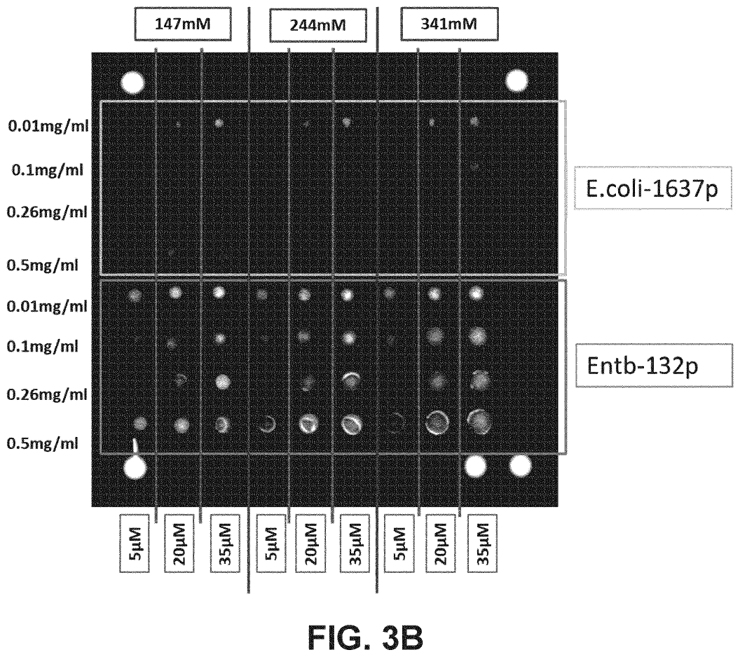

A Cy3 fluorescence image of the three-dimensional networks without any bound analyte is shown in FIG. 3A and a Cy5 fluorescence image of the three-dimensional networks with Cy5 labeled analyte molecules is shown in FIG. 3B. Three-dimensional networks made using 341 mM buffer with 20 μM and 35 μM probe concentrations were observed by visual inspection to have more homogeneous spot morphologies compared to other three-dimensional networks. For example, three-dimensional networks made using 341 mM buffer with 20 μM and 35 μM probe concentrations generally showed less of a halo effect, which, without being bound by theory, is believed to be an indication that a three-dimensional network is not completely homogeneous.

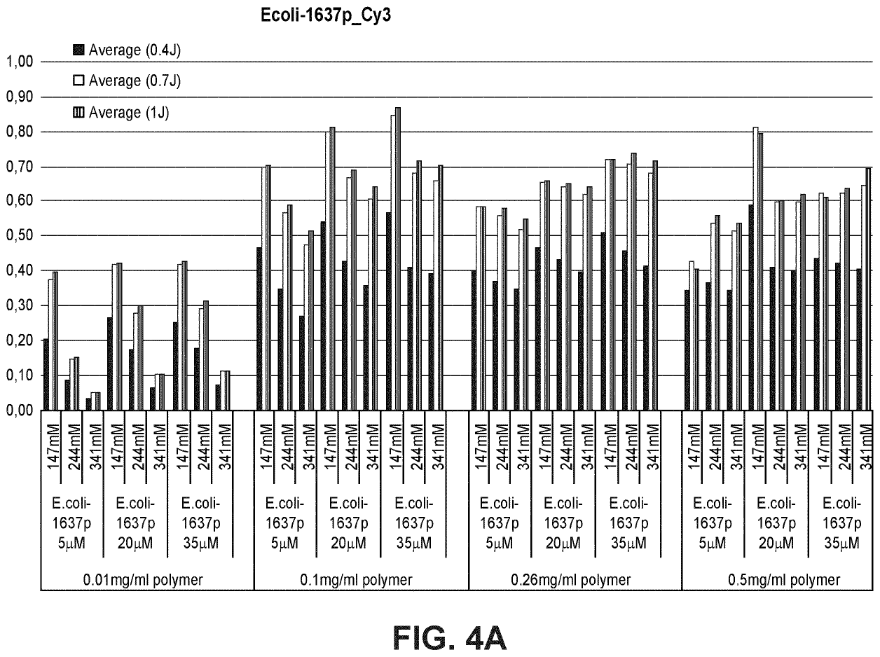

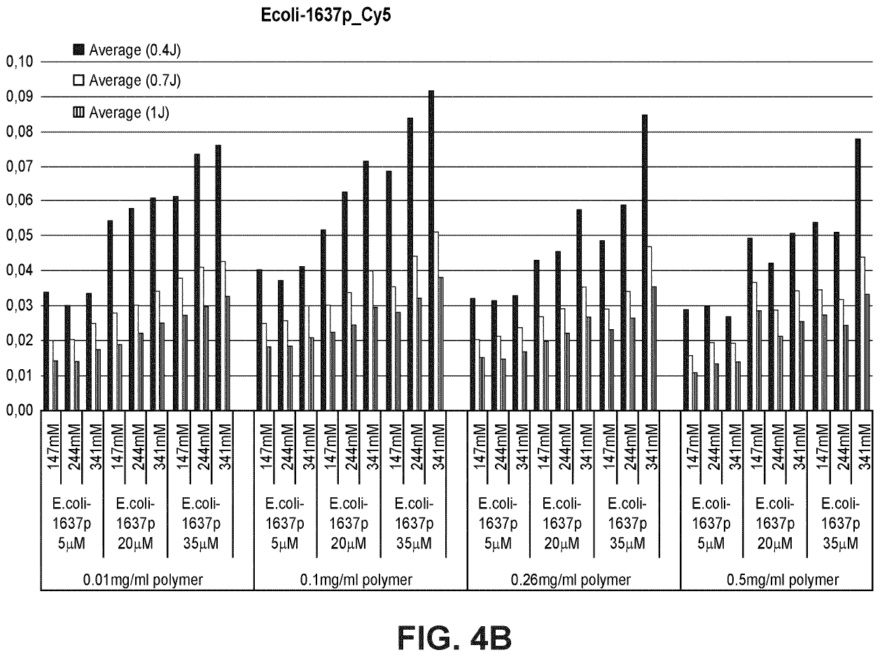

Cy3 signal intensities for probe E. coli-1637p are plotted in FIG. 4A. Three-dimensional networks made with 0.01 mg/ml polymer concentration showed relatively low signal intensities whereas 0.1 mg/ml, 0.26 mg/ml and 0.5 mg/ml polymer concentrations showed similar, higher signals. Three-dimensional networks made with 5 μM oligonucleotide concentration generally showed the lowest signal, while three-dimensional networks made with 20 μM and 35 μM showed similar signals. Three-dimensional networks made with 244 mM and 341 mM buffer concentration showed similar signal intensities at a respective oligonucleotide concentration. Increased cross-linking energy was found to increase the signal intensities.

Cy5 signal intensities for probe E. coli-1637p are shown in FIG. 4B. Three-dimensional networks made with 5 μM oligonucleotide concentration showed the lowest signals and 35 μM showed slightly higher signals than 20 μM. Three-dimensional networks made with 341 mM buffer concentration at a respective oligonucleotide concentration showed the highest signal intensities. Increased cross-linking energy was found to decrease the signal intensities.

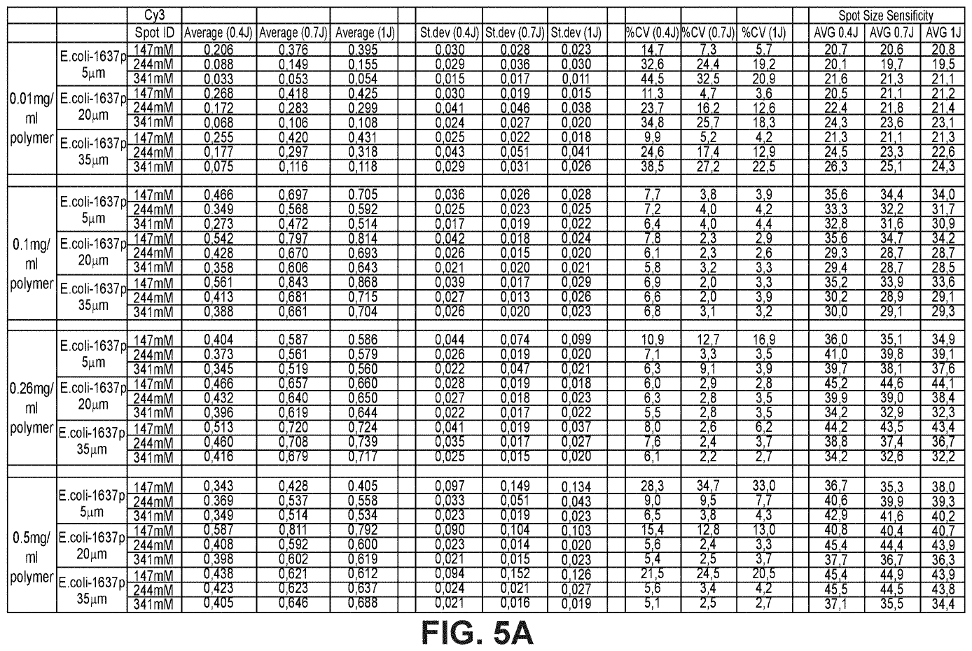

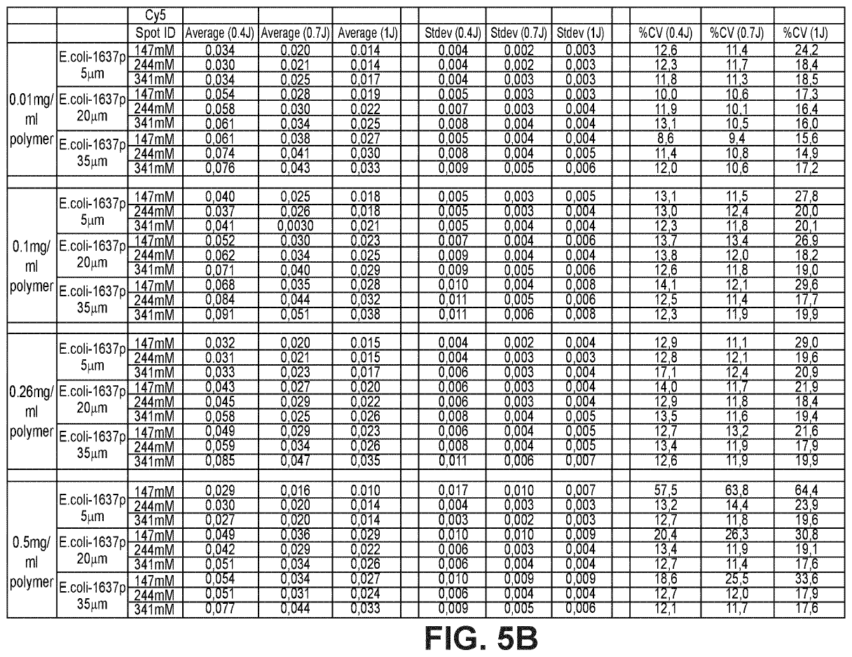

Coefficients of variation, which provide a measure of three-dimensional network homogeneity, for the three-dimensional networks with the E. coli-1637p probe are shown in FIG. 5A (Cy3) and FIG. 5B (Cy5). As shown in FIG. 5A, three-dimensional networks made with 0.01 mg/ml polymer showed high % CVs, while 0.7 J/cm² of cross-linking energy showed better % CVs than 1 J/cm². As shown in FIG. 5B, three-dimensional networks made with 1 J/cm² showed relatively high % CVs, while three-dimensional networks made with 5 μM oligonucleotide had relatively high % CVs with all combinations of parameters. While three-dimensional networks made with 0.4 J/cm² of cross-linking energy showed good signal intensities, comparatively better % CVs were observed with three-dimensional networks made with 0.7 J/cm² of cross-linking energy.

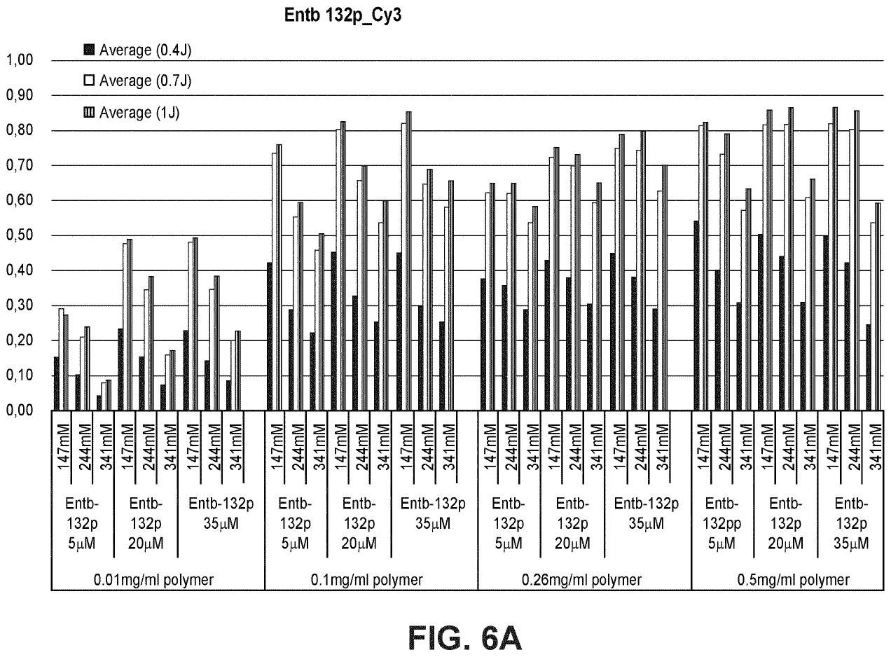

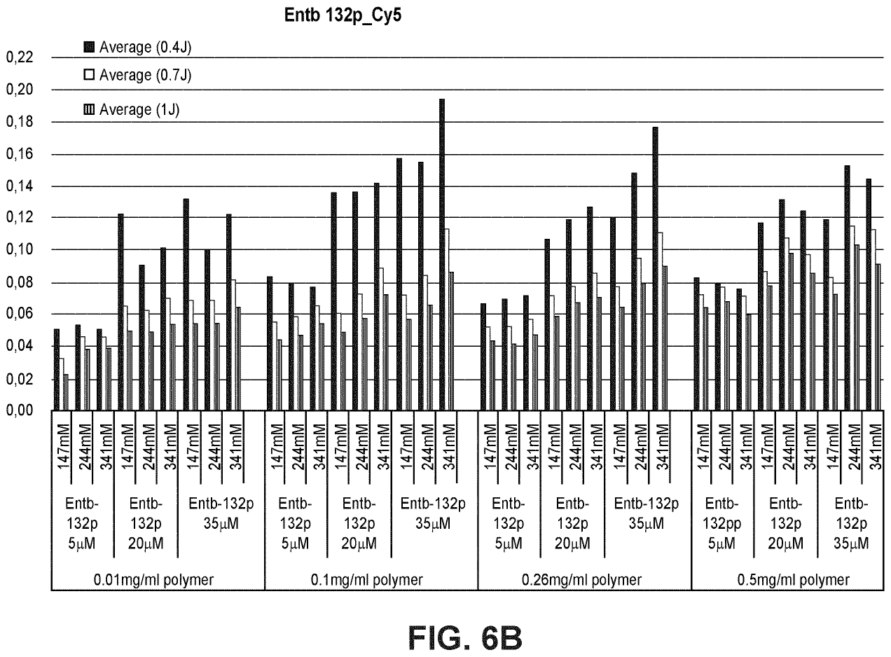

Cy3 signal intensities for probe Entb-132p are plotted in FIG. 6A. A similar profile to FIG. 4A was observed.

Cy5 signal intensities for probe Entb-132p are shown in FIG. 6B. Three-dimensional networks made with 0.01 mg/ml polymer concentration shows relativley low signal intensities whereas 0.1 mg/ml, 0.26 mg/ml and 0.5 mg/ml polymer concentrations showed similar, higher signals. Three-dimensional networks made with 5 μM oligonucleotide concentration showed the lowest signals and 35 μM showed slightly higher signals than 20 μM. Three-dimensional networks made with 341 mM buffer concentration at a respective oligonucleotide concentration showed highest signal intensities. Increased cross-linking energy decreased the signal intensities, with 0.4 J/cm² showing relatively high signal intensities.

Coefficients of variation for the three-dimensional networks with the Entb-132p probe are shown in FIG. 7A (Cy3) and FIG. 7B (Cy5). The profile shown in FIG. 7A is similar to the profile shown in FIG. 5A. While three-dimensional networks made with 1 J/cm² of cross-linking energy showed good signal intensities, comparatively better % CVs were observed with three-dimensional networks made with 0.4 J/cm² and 0.7 J/cm² of cross-linking energy.

### 6.3. Example 3: Comparison of Three-Dimensional Networks Made with Different Parameters

Following the studies of Example 1 and Example 2, arrays made using the following parameters were compared for array functionality and production run yield.

TABLE 2

| Factor | Original | | New | |
|---|---|---|---|---|
| Polymer concentration | 1 | mg/ml | 0.1 | mg/ml |
| Phosphate concentration | 341 | mM | 250 | mM |
| Oligonucleotide probe | 20 | μM | 20 | μM |
| concentration | | | | |
| Cross-linking energy | 1 | J/cm² | 0.7 | J/cm² |

The functionality of the arrays made using the original and new parameters was comparable, while the yield of the production runs was better with the new process parameters (data not shown).

### 7. SPECIFIC EMBODIMENTS

The present disclosure is exemplified by the specific embodiments below.
1. A process for making a three-dimensional hydrogel network, comprising:
   (a) exposing a mixture (optionally positioned on the surface of a substrate) to salt crystal forming conditions, the mixture comprising:
      (i) an aqueous salt solution,
      (ii) water-soluble polymer chains,
      (iii) cross-linker moieties, and
      (iv) optionally, probe molecules,
      thereby forming a mixture containing one or more salt crystals;
   (b) cross-linking the water-soluble polymer chains in the mixture containing one or more salt crystals, thereby forming a hydrogel containing one or more salt crystals; and
   (c) contacting the hydrogel containing one or more salt crystals with a solvent in which the one or more salt crystals are soluble, thereby dissolving the salt crystals;
thereby forming the three-dimensional hydrogel network,
optionally wherein:
   A) the concentration of the water-soluble polymer chains in the mixture of step (a) is such that precipitation of water-soluble polymer chains from the mixture does not occur before formation of the one or more salt crystals in step (a); and/or
   B) the concentration of the water-soluble polymer chains in the mixture of step (a) is such that the water-soluble polymer chains and salt crystals co-precipitate in step (a); and/or
   C) the concentration of the water-soluble polymer chains in the mixture of step (a) is less than 1 mg/ml.
2. The process of embodiment 1, wherein the concentration of the water-soluble polymer chains in the mixture of step (a) is such that precipitation of water-soluble polymer chains from the mixture does not occur before formation of the one or more salt crystals in step (a).
3. The process of embodiment 1 or embodiment 2, wherein the concentration of the water-soluble polymer chains in the mixture of step (a) is such that the water-soluble polymer chains and salt crystals co-precipitate during step (a).
4. The process of any one of embodiments 1 to 3, wherein the concentration of the water-soluble polymer chains in the mixture of step (a) is less than 1 mg/ml.
5. The process of any one of embodiments 1 to 4, wherein the concentration of the water-soluble polymer chains in the mixture of step (a) ranges from a lower limit (“polymer lower limit”) that is at least 0.01 mg/ml to an upper limit (“polymer upper limit”) that is less than 1 mg/ml.
6. The process of embodiment 5, wherein the polymer lower limit is 0.01 mg/ml.
7. The process of embodiment 5, wherein the polymer lower limit is 0.05 mg/ml.
8. The process of embodiment 5, wherein the polymer lower limit is 0.1 mg/ml.
9. The process of any one of embodiments 5 to 8, wherein the polymer upper limit is 0.5 mg/ml.
10. The process of any one of embodiments 5 to 8, wherein the polymer upper limit is 0.4 mg/ml.
11. The process of any one of embodiments 5 to 8, wherein the polymer upper limit is 0.3 mg/ml.
12. The process of any one of embodiments 5 to 8, wherein the polymer upper limit is 0.2 mg/ml.
13. The process of any one of embodiments 5 to 8, wherein the polymer upper limit is 0.1 mg/ml.
14. The process of embodiment 5, wherein the concentration of the water-soluble polymer chains in the mixture of step (a) is 0.1 mg/ml.
15. The process of any one of embodiments 1 to 14, wherein the cross-linker moieties are covalently attached to the water-soluble polymer chains.
16. The process of any one of embodiments 1 to 15, wherein the cross-linker moieties are photoreactive.
17. The process of embodiment 16, wherein the cross-linking comprises exposing the mixture containing one or more salt crystals to UV light.
18. The process of embodiment 17, wherein the cross-linking comprises exposing the mixture containing one or more salt crystals to UV light having a wavelength of 254 nm.
19. The process of any one of embodiments 1 to 18, wherein the cross-linker moieties are photoreactive and step (b) comprises cross-linking the water-soluble polymer chains with a UV light energy dose of less than 1 J/cm².
20. The process of embodiment 19, wherein step (b) comprises cross-linking the water-soluble polymer chains with a UV light energy dose that ranges from a lower limit (“cross-linking energy lower limit”) that is at least 0.4 J/cm² to an upper limit (“cross-linking energy upper limit”) that is less than 1 J/cm².
21. The process of embodiment 20, wherein the cross-linking energy lower limit is 0.4 J/cm².
22. The process of embodiment 20, wherein the cross-linking energy lower limit is 0.5 J/cm².
23. The process of embodiment 20, wherein the cross-linking energy lower limit is 0.6 J/cm².
24. The process of embodiment 20, wherein the cross-linking energy lower limit is 0.7 J/cm².
25. The process of any one of embodiments 20 to 24, wherein the cross-linking energy upper limit is 0.9 J/cm².
26. The process of any one of embodiments 20 to 24, wherein the cross-linking energy upper limit is 0.8 J/cm².
27. The process of any one of embodiments 20 to 24, wherein the cross-linking energy upper limit is 0.7 J/cm².
28. The process of embodiment 20, wherein step (b) comprises cross-linking the water-soluble polymer chains with a UV energy dose of 0.7 J/cm².
29. The process of any one of embodiments 1 to 28, wherein the aqueous salt solution comprises phosphate ions.
30. The process of embodiment 29, wherein the concentration of the phosphate ions in the mixture of step (a) ranges from a lower limit (“phosphate lower limit”) that is at least 125 mM to an upper limit (“phosphate upper limit”) that is less than 350 mM.
31. The process of embodiment 30, wherein the phosphate lower limit is 125 mM.
32. The process of embodiment 30, wherein the phosphate lower limit is 150 mM.
33. The process of embodiment 30, wherein the phosphate lower limit is 200 mM.
34. The process of embodiment 30, wherein the phosphate lower limit is 225 mM.
35. The process of any one of embodiments 30 to 34, wherein the phosphate upper limit is 340 mM.
36. The process of any one of embodiments 30 to 34, wherein the phosphate upper limit is 300 mM.
37. The process of any one of embodiments 30 to 34, wherein the phosphate upper limit is 250 mM.
38. The process of embodiment 30, wherein the concentration of phosphate ions in the mixture of step (a) is 250 mM.
39. The process of any one of embodiments 1 to 38, wherein the aqueous salt solution comprises a sodium phosphate solution.
40. The process of embodiment 39, wherein the aqueous salt solution comprises a solution produced by a process comprising dissolving disodium hydrogen phosphate, sodium dihydrogen phosphate or a combination thereof in water or an aqueous solution.
41. The process of any one of embodiments 1 to 40, wherein the salt crystal forming conditions result in formation one or more needle-shaped crystals such that one or more long channels are produced after dissolution of the salt crystals.
42. The process of any one of embodiments 1 to 28, wherein the mixture of step (a) comprises at least two types of monovalent metal ions having a total concentration of at least 500 mM.
43. The process of embodiment 42, wherein the mixture of step (a) comprises at least two types of monovalent metal ions having a total concentration of 500 mM to 1000 mM.
44. The process of embodiment 43, wherein total concentration of monovalent metal ions in the mixture of step (a) is 550 mM to 800 mM.
45. The process of embodiment 44, wherein total concentration of monovalent metal ions in the mixture of step (a) is 600 mM to 750 mM.
46. The process of any one of embodiments 42 to 45, wherein the mixture of step (a) comprises two types of monovalent metal ions.
47. The process of embodiment 46, wherein the concentration of each of the two monovalent ion is at least 150 mM or at least 200 mM.
48. The process of embodiment 46 or embodiment 47, wherein the monovalent metal ions are selected from sodium ions, potassium ions, and lithium ions.
49. The process of embodiment 46 or embodiment 47, wherein the monovalent metal ions are sodium ions and potassium ions.
50. The process of embodiment 49, wherein concentration of sodium ions in the mixture of step (a) is at least 300 mM.
51. The process of embodiment 50, wherein the concentration of sodium ions in the mixture of step (a) is 300 mM to 500 mM.
52. The process of embodiment 51, wherein the concentration of sodium ions in the mixture of step (a) is 300 mM to 400 mM.
53. The process of embodiment 52, wherein the concentration of sodium ions in the mixture of step (a) is 350 mM.
54. The process of any one of embodiments 49 to 53, wherein the concentration of potassium ions in the mixture of step (a) is 150 mM to 500 mM.
55. The process of embodiment 54, wherein the concentration of potassium ions in the mixture of step (a) is 175 mM to 400 mM.
56. The process of embodiment 55, wherein the concentration of potassium ions in the mixture of step (a) is 200 mM to 350 mM.
57. The process of embodiment 56, wherein the concentration of potassium ions in the mixture of step (a) is 250 mM to 350 mM.
58. The process of any one of embodiments 42 to 45, wherein the mixture of step (a) comprises three types of monovalent metal ions.
59. The process of embodiment 58, wherein the concentration of at least two of the monovalent ions is at least 150 mM each or at least 200 mM each.
60. The process of embodiment 58 or embodiment 59, wherein the monovalent metal ions are sodium ions, potassium ions, and lithium ions.
61. The process of embodiment 60, wherein the concentration of sodium ions in the mixture of step (a) is at least 250 mM.
62. The process of embodiment 61, wherein the concentration of sodium ions in the mixture of step (a) is 250 mM to 500 mM.
63. The process of embodiment 62, wherein the concentration of sodium ions in the mixture of step (a) is 300 mM to 400 mM.
64. The process of embodiment 63, wherein the concentration of sodium ions in the mixture of step (a) is 350 mM.
65. The process of any one of embodiments 60 to 64, wherein the concentration of potassium ions in the mixture of step (a) is 150 mM to 500 mM.
66. The process of embodiment 65, wherein the concentration of potassium ions in the mixture of step (a) is 200 mM to 400 mM.
67. The process of embodiment 66, wherein the concentration of potassium ions in the mixture of step (a) is 250 mM to 350 mM.
68. The process of any one of embodiments 42 to 67, wherein the concentration of phosphate ions in the mixture is at least 250 mM.
69. The process of embodiment 68, wherein the concentration of phosphate ions in the mixture is 250 mM to 1000 mM.
70. The process of embodiment 69, wherein the concentration of phosphate ions in the mixture is 550 mM to 800 mM.
71. The process of embodiment 70, wherein the concentration of phosphate ions in the mixture is 600 mM to 750 mM.
72. The process of any one of embodiments 42 to 71, wherein the salt crystal forming conditions result in formation one or more needle-shaped crystals such that one or more long channels are produced after dissolution of the salt crystals.
73. The process of any one of embodiments 42 to 72, wherein the salt crystal forming conditions result in formation of one or more compact crystals such that one or more short channels are produced after dissolution of the salt crystals.
74. The process of any one of embodiments 1 to 73, wherein the water-soluble polymer chains comprise homopolymer chains.
75. The process of any one of embodiments 1 to 73, wherein the water-soluble polymer chains comprise copolymer chains.
76. The process of any one of embodiments 1 to 73, wherein the water-soluble polymer chains comprise a mixture of homopolymer and copolymer chains.
77. The process of any one of embodiments 74 to 76, wherein the water-soluble polymer chains comprise polymer chains polymerized from one or more species of monomers.
78. The process of embodiment 77, wherein each species of monomer comprises a polymerizable group independently selected from an acrylate group, a methacrylate group, an ethacrylate group, a 2-phenyl acrylate group, an acrylamide group, a methacrylamide group, an itaconate group, and a styrene group.
79. The process of embodiment 78, wherein at least one monomer species in the water-soluble polymer comprises a methacrylate group.
80. The process of embodiment 79, wherein the at least one monomer species comprising a methacrylate group is methacryloyloxybenzophenone (MABP).
81. The process of any one of embodiment 77, wherein the water-soluble polymer comprises a polymer polymerized from dimethylacrylamide (DMAA), methacryloyloxybenzophenone (MABP), and sodium 4-vinylbenzenesulfonate (SSNa).
82. The process of embodiment 81, wherein the water-soluble polymer comprises a polymer polymerized from dimethylacrylamide (DMAA), methacryloyloxybenzophenone (MABP), and sodium 4-vinylbenzenesulfonate (SSNa) and comprising 2.5 to 7.5 mol % MABP,
2 to 5 mol % SSNa, and the balance DMAA.
83. The process of embodiment 81, wherein the water-soluble polymer comprises a polymer polymerized from dimethylacrylamide (DMAA), methacryloyloxybenzophenone (MABP), and sodium 4-vinylbenzenesulfonate (SSNa) in a DMAA:MABP:SSNa molar ratio of 92.5:5:2.5.
84. The process of any one of embodiments 1 to 83, wherein the water-soluble polymer chains are chains of a copolymer comprising the cross-linker moieties.
85. The process of embodiment 84, wherein the water-soluble polymer chains comprise at least two cross-linker moieties per polymer molecule.
86. The process of any one of embodiments 1 to 85, wherein the cross-linker moieties are selected from benzophenone, a thioxanthone, a benzoin ether, ethyl eosin, eosin Y, rose bengal, camphorquinone, erythirosin, 4,4′ azobis(4-cyanopentanoic) acid, 2,2-azobis[2-(2-imidazolin-2-yl) propane]dihydrochloride, and benzoyl peroxide.
87. The process of embodiment 86, wherein the cross-linker moieties are benzophenone moieties.
88. The process of any one of embodiments 1 to 87, wherein the average molecular weight of the water-soluble polymer chains is at least 100 kDa.
89. The process of embodiment 88, wherein the average molecular weight of the water-soluble polymer chains is at least 200 kDa.
90. The process of embodiment 88, wherein the average molecular weight of the water-soluble polymer chains is at least 300 kDa.
91. The process of embodiment 88, wherein the average molecular weight of the water-soluble polymer chains is at least 400 kDa.
92. The process of any one of embodiments 88 to 91, wherein the average molecular weight of the water-soluble polymer chains is no more than 600 kDa.
93. The process of any one of embodiments 88 to 91, wherein the average molecular weight of the water-soluble polymer chains is no more than 500 kDa.
94. The process of any one of embodiments 88 to 90, wherein the average molecular weight of the water-soluble polymer chains is no more than 400 kDa.
95. The process of any one of embodiments 88 to 90, wherein the average molecular weight of the water-soluble polymer chains is no more than 300 kDa.
96. The process of any one of embodiments 1 to 87, wherein the average molecular weight of the water-soluble polymer chains is 200 kDa to 400 kDa.
97. A process for making a three-dimensional hydrogel network, comprising:
   (a) exposing a mixture (optionally positioned on the surface of a substrate) to salt crystal forming conditions, the mixture comprising:
      (i) an aqueous salt solution comprising phosphate ions at a concentration ranging from a lower limit (“phosphate lower limit”) that is at least 125 mM to an upper limit (“phosphate upper limit”) that is less than 350 mM
      (ii) water-soluble polymer chains at a concentration of less than 1 mg/ml, wherein the water-soluble polymer chains comprise a polymer polymerized from dimethylacrylamide (DMAA), methacryloyloxybenzophenone (MABP), and sodium 4-vinylbenzenesulfonate (SSNa), and
      (iii) optionally, probe molecules,
      thereby forming a mixture containing one or more salt crystals;
   (b) cross-linking the water-soluble polymer chains in the mixture containing one or more salt crystals, thereby forming a hydrogel containing one or more salt crystals; and
   (c) contacting the hydrogel containing one or more salt crystals with a solvent in which the one or more salt crystals are soluble, thereby dissolving the salt crystals;
thereby forming the three-dimensional hydrogel network.
98. The process of embodiment 97, wherein the concentration of the water-soluble polymer chains in the mixture of step (a) ranges from a lower limit (“polymer lower limit”) that is at least 0.01 mg/ml to an upper limit (“polymer upper limit”) that is less than 1 mg/ml.
99. The process of embodiment 98, wherein the polymer lower limit is 0.01 mg/ml.
100. The process of embodiment 98, wherein the polymer lower limit is 0.05 mg/ml.
101. The process of embodiment 98, wherein the polymer lower limit is 0.1 mg/ml.
102. The process of any one of embodiments 98 to 101, wherein the polymer upper limit is 0.5 mg/ml.
103. The process of any one of embodiments 98 to 101, wherein the polymer upper limit is 0.4 mg/ml.
104. The process of any one of embodiments 98 to 101, wherein the polymer upper limit is 0.3 mg/ml.
105. The process of any one of embodiments 98 to 101, wherein the polymer upper limit is 0.2 mg/ml.
106. The process of any one of embodiments 98 to 101, wherein the polymer upper limit is 0.1 mg/ml.
107. The process of embodiment 97, wherein the concentration of the water-soluble polymer chains in the mixture of step (a) is 0.1 mg/ml.
108. The process of any one of embodiments 97 to 107, wherein the phosphate lower limit is 125 mM.
109. The process of any one of embodiments 97 to 107, wherein the phosphate lower limit is 150 mM.
110. The process of any one of embodiments 97 to 107, wherein the phosphate lower limit is 200 mM.
111. The process of any one of embodiments 97 to 107, wherein the phosphate lower limit is 225 mM.
112. The process of any one of embodiments 97 to 111, wherein the phosphate upper limit is 340 mM.
113. The process of any one of embodiments 97 to 111, wherein the phosphate upper limit is 300 mM.
114. The process of any one of embodiments 97 to 111, wherein the phosphate upper limit is 250 mM.
115. The process of any one of embodiments 97 to 107, wherein the concentration of phosphate ions in the mixture of step (a) is 250 mM.
116. The process of any one of embodiments 97 to 115, wherein the aqueous salt solution comprises a sodium phosphate solution.
117. The process of embodiment 116, wherein the aqueous salt solution comprises a solution produced by a process comprising dissolving disodium hydrogen phosphate, sodium dihydrogen phosphate or a combination thereof in water or an aqueous solution.
118. The process of any one of embodiments 97 to 117, wherein the cross-linking comprises exposing the mixture containing one or more salt crystals to UV light.
119. The process of embodiment 118, wherein the cross-linking comprises exposing the mixture containing one or more salt crystals to UV light having a wavelength of 254 nm.
120. The process of any one of embodiments 97 to 119, wherein step (b) comprises cross-linking the water-soluble polymer chains with a UV light energy dose of less than 1 J/cm².
121. The process of embodiment 120, wherein step (b) comprises cross-linking the water-soluble polymer chains with a UV light energy dose that ranges from a lower limit (“cross-linking energy lower limit”) that is at least 0.4 J/cm² to an upper limit (“cross-linking energy upper limit”) that is less than 1 J/cm².
122. The process of embodiment 121, wherein the cross-linking energy lower limit is 0.4 J/cm².
123. The process of embodiment 121, wherein the cross-linking energy lower limit is 0.5 J/cm².
124. The process of embodiment 121, wherein the cross-linking energy lower limit is 0.6 J/cm².
125. The process of embodiment 121, wherein the cross-linking energy lower limit is 0.7 J/cm².
126. The process of any one of embodiments 121 to 125, wherein the cross-linking energy upper limit is 0.9 J/cm².
127. The process of any one of embodiments 121 to 125, wherein the cross-linking energy upper limit is 0.8 J/cm².
128. The process of any one of embodiments 121 to 125, wherein the cross-linking energy upper limit is 0.7 J/cm².
129. The process of embodiment 120, wherein step (b) comprises cross-linking the water-soluble polymer chains with a UV energy dose of 0.7 J/cm².
130. The process of any one of embodiments 97 to 129, wherein the water-soluble polymer comprises a polymer polymerized from dimethylacrylamide (DMAA), methacryloyloxybenzophenone (MABP), and sodium 4-vinylbenzenesulfonate (SSNa) and comprising 2.5 to 7.5 mol % MABP, 2 to 5 mol % SSNa, and the balance DMAA.
131. The process of any one of embodiments 97 to 129, wherein the water-soluble polymer comprises a polymer polymerized from dimethylacrylamide (DMAA), methacryloyloxybenzophenone (MABP), and sodium 4-vinylbenzenesulfonate (SSNa) in a DMAA:MABP:SSNa molar ratio of 92.5:5:2.5.
132. The process of any one of embodiments 97 to 131, wherein the average molecular weight of the water-soluble polymer chains is at least 100 kDa.
133. The process of embodiment 132, wherein the average molecular weight of the water-soluble polymer chains is at least 200 kDa.
134. The process of embodiment 132, wherein the average molecular weight of the water-soluble polymer chains is at least 300 kDa.
135. The process of embodiment 132, wherein the average molecular weight of the water-soluble polymer chains is at least 400 kDa.
136. The process of any one of embodiments 132 to 135, wherein the average molecular weight of the water-soluble polymer chains is no more than 600 kDa.
137. The process of any one of embodiments 132 to 135, wherein the average molecular weight of the water-soluble polymer chains is no more than 500 kDa.
138. The process of any one of embodiments 132 to 135, wherein the average molecular weight of the water-soluble polymer chains is no more than 400 kDa.
139. The process of any one of embodiments 132 to 135, wherein the average molecular weight of the water-soluble polymer chains is no more than 300 kDa.
140. The process of any one of embodiments 97 to 118, wherein the average molecular weight of the water-soluble polymer chains is 200 kDa to 400 kDa.
141. The process of any one of embodiments 1 to 140, wherein the salt crystal forming conditions comprise dehydrating the mixture.
142. The process of embodiment 141, which comprises dehydrating the mixture by heating the mixture, exposing the mixture to a vacuum, reducing the humidity of the atmosphere surrounding the mixture, or a combination thereof.
143. The process of embodiment 142, which comprises dehydrating the mixture by exposing the mixture to a vacuum.
144. The process of embodiment 142, which comprises dehydrating the mixture by heating the mixture.
145. The process of embodiment 144, wherein heating the mixture comprises contacting the mixture with a gas that has a temperature which is higher than the temperature of the mixture.
146. The process of any one of embodiments 1 to 141, wherein the salt crystal forming conditions comprise cooling the mixture until the mixture becomes supersaturated with a salt.
147. The process of embodiment 146, which comprises cooling the mixture by contacting the mixture with a gas that has a temperature which is lower than the temperature of the mixture.
148. The process of any one of embodiments 1 to 147, wherein the temperature of the mixture during step (a) is maintained above the dew point of the atmosphere surrounding the mixture.
149. The process of any one of embodiments 1 to 148, wherein the aqueous salt solution has a pH ranging from 6 to 9.
150. The process of embodiment 149, wherein the aqueous salt solution has a pH ranging from 7 to 8.5.
151. The process of embodiment 150, wherein the aqueous salt solution has a pH of 8.
152. The process of any one of embodiments 1 to 151, which further comprises, prior to step (a), forming the mixture
153. The process of any one of embodiments 1 to 152, wherein the solvent is water or a water-based buffer.
154. The process of embodiment 153, wherein the solvent is water.
155. The process of embodiment 153, wherein the solvent is a water-based buffer.
156. The process of embodiment 155, wherein the water-based buffer comprises phosphate, methanol, ethanol, propanol, or a mixture thereof.
157. The process of any one of embodiments 1 to 156, wherein the mixture of step (a) further comprises probe molecules.
158. The process of embodiment 157, wherein at least some, the majority or all the probe molecules comprise a nucleic acid, a nucleic acid derivative, a peptide, a polypeptide, a protein, a carbohydrate, a lipid, a cell, a ligand, or a combination thereof.
159. The process of embodiment 158, wherein at least some of the probe molecules comprise a nucleic acid or a nucleic acid derivative.
160. The process of embodiment 158, wherein at least a majority of the probe molecules comprise a nucleic acid or a nucleic acid derivative.
161. The process of embodiment 158, wherein all the probe molecules comprise a nucleic acid or a nucleic acid derivative.
162. The process of embodiment 157, wherein at least some, the majority or all the probe molecules comprise an antibody, an antibody fragment, an antigen, an epitope, an enzyme, an enzyme substrate, an enzyme inhibitor, a nucleic acid, or a combination thereof.
163. The process of embodiment 162, wherein at least some of the probe molecules comprise a nucleic acid.
164. The process of embodiment 162, wherein at least a majority of the probe molecules comprise a nucleic acid.
165. The process of embodiment 162, wherein all the probe molecules comprise a nucleic acid.
166. The process of any one of embodiments 163 to 165, wherein the nucleic acid is an oligonucleotide.
167. The process of embodiment 166, wherein the oligonucleotide is 12 to 30 nucleotides long.
168. The process of embodiment 166, wherein the oligonucleotide is 14 to 30 nucleotides long.
169. The process of embodiment 166, wherein the oligonucleotide is 14 to 25 nucleotides long.
170. The process of embodiment 166, wherein the oligonucleotide is 14 to 20 nucleotides long.
171. The process of embodiment 166, wherein the oligonucleotide is 15 to 30 nucleotides long.
172. The process of embodiment 166, wherein the oligonucleotide is 15 to 25 nucleotides long.
173. The process of embodiment 166, wherein the oligonucleotide is 15 to 20 nucleotides long.
174. The process of embodiment 166, wherein the oligonucleotide is 16 to 30 nucleotides long.
175. The process of embodiment 166, wherein the oligonucleotide is 16 to 25 nucleotides long.
176. The process of embodiment 166, wherein the oligonucleotide is 16 to 20 nucleotides long.
177. The process of embodiment 166, wherein the oligonucleotide is 15 to 40 nucleotides long.
178. The process of embodiment 166, wherein the oligonucleotide is 15 to 45 nucleotides long.
179. The process of embodiment 166, wherein the oligonucleotide is 15 to 50 nucleotides long.
180. The process of embodiment 166, wherein the oligonucleotide is 15 to 60 nucleotides long.
181. The process of embodiment 166, wherein the oligonucleotide is 20 to 55 nucleotides long.
182. The process of embodiment 166, wherein the oligonucleotide is 18 to 60 nucleotides long.
183. The process of embodiment 166, wherein the oligonucleotide is 20 to 50 nucleotides long.
184. The process of embodiment 166, wherein the oligonucleotide is 30 to 90 nucleotides long.
185. The process of embodiment 166, wherein the oligonucleotide is 20 to 100 nucleotides long.
186. The process of embodiment 166, wherein the oligonucleotide is 20 to 120 nucleotides long.
187. The process of embodiment 166, wherein the oligonucleotide is 20 to 40 nucleotides long.
188. The process of embodiment 166, wherein the oligonucleotide is 20 to 60 nucleotides long.
189. The process of embodiment 166, wherein the oligonucleotide is 40 to 80 nucleotides long.
190. The process of embodiment 166, wherein the oligonucleotide is 40 to 100 nucleotides long.
191. The process of embodiment 166, wherein the oligonucleotide is 40 to 60 nucleotides long.
192. The process of embodiment 166, wherein the oligonucleotide is 60 to 80 nucleotides long.
193. The process of embodiment 166, wherein the oligonucleotide is 80 to 100 nucleotides long.
194. The process of embodiment 166, wherein the oligonucleotide is 100 to 120 nucleotides long.
195. The process of embodiment 166, wherein the oligonucleotide is 12 to 150 nucleotides long.
196. The process of any one of embodiments 166 to 195, wherein the concentration of the oligonucleotide in the mixture of step (a) ranges from 5 μM to 35 μM.
197. The process of embodiment 196, wherein the concentration of the oligonucleotide in the mixture of step (a) is 15 μM to 25 μM.
198. The process of embodiment 196, wherein the concentration of the oligonucleotide in the mixture of step (a) is 5 μM.
199. The process of embodiment 196, wherein the concentration of the oligonucleotide in the mixture of step (a) is 20 μM.
200. The process of embodiment 196, wherein the concentration of the oligonucleotide in the mixture of step (a) is 35 μM.
201. The process of any one of embodiments 1 to 200, further comprising, prior to step (a), a step of applying the mixture to a surface of a substrate.
202. The process of embodiment 201, wherein the mixture is applied in a volume of 100 pl to 5 nl.
203. The process of embodiment 201, wherein the mixture is applied in a volume of 100 pl to 1 nl.
204. The process of embodiment 201, wherein the mixture is applied in a volume of 200 pl to 1 nl.
205. The process of embodiment 201, wherein the mixture is applied in a volume of 1 nl to 2 nl.
206. The process of embodiment 201, wherein the mixture is applied in a volume of 1.5 nl to 2 nl.
207. The process of any one of embodiments 201 to 206, wherein the step of applying the mixture to the substrate comprises spraying the mixture onto the surface of the substrate.
208. The process of embodiment 207, wherein the mixture is sprayed by an inkjet printer.
209. The process of any one of embodiments 201 to 208, wherein the substrate comprises an organic polymer or an inorganic material having a self-assembled monolayer of organic molecules on the surface.
210. The process of embodiment 209, wherein the substrate comprises an organic polymer.
211. The process of embodiment 210, wherein the organic polymer is selected from cycloolefin copolymers, polystyrene, polyethylene, polypropylene, polycarbonate, and polymethylmethacrylate.
212. The process of embodiment 211, wherein the substrate comprises polymethylmethacrylate, polystyrene, or cycloolefin copolymers.
213. The process of embodiment 211, wherein the substrate comprises polystyrene.
214. The process of embodiment 209, wherein the substrate comprises an inorganic material having an alkyl silane self-assembled monolayer on the surface.
215. The process of any one of embodiments 201 to 214, wherein the substrate comprises a microwell plate.
216. The process of any one of embodiments 201 to 215, wherein the polymer is cross-linked to the surface in step (b).
217. The process of embodiment 216, in which a water-swellable polymer is produced that is cross-linked to the surface.
218. The process of embodiment 217, wherein the water-swellable polymer can absorb up to 50 times its weight of deionized, distilled water.
219. The process of embodiment 217 or embodiment 218, wherein the water-swellable polymer can absorb 5 to 50 times its own volume of deionized, distilled water.
220. The process of any one of embodiments 217 to 219, wherein the water-swellable polymer can absorb up to 30 times its weight of saline.
221. The process of any one of embodiments 217 to 220, wherein the water-swellable polymer can absorb 4 to 30 times its own volume of saline.
222. A process for making an array, comprising generating a plurality of three-dimensional hydrogel networks by the process of any one of embodiments 1 to 221 at discrete spots on the surface of the same substrate.
223. The process of embodiment 222, wherein the three-dimensional hydrogel networks are generated simultaneously.
224. The process of embodiment 222, wherein the three-dimensional hydrogel networks are generated sequentially.
225. The process of any one of embodiments 222 to 224, further comprising cross-linking the plurality of three-dimensional hydrogel networks to the surface of the substrate.
226. A process for making an array, comprising positioning a plurality of three-dimensional hydrogel networks produced or obtainable according to the process of any one of embodiments 1 to 221 at discrete spots on a surface of the same substrate.
227. The process of any one of embodiments 222 to 226, further comprising cross-linking the plurality of three-dimensional hydrogel networks to the surface.
228. A process for making an array, comprising positioning a plurality of three-dimensional hydrogel networks produced or obtainable according to the process of any one of embodiments 196 to 221 at discrete spots on a surface of the same substrate.
229. The process of embodiment 228, wherein the positioning comprises applying the mixtures from which the three-dimensional hydrogel networks are formed at the discrete spots.
230. The process of any one of embodiments 222 to 229, wherein the spots are arranged in columns and/or rows.
231. A three-dimensional hydrogel network produced or obtainable by the process of any one of embodiments 1 to 221.
232. A plurality of three-dimensional hydrogel networks each having a surface and an interior comprising (a) a cross-linked polymer, (b) one or more channels, and (c) probe molecules immobilized in the network, wherein when contacted with an analyte capable of binding to the probe molecules to produce a signal, the measured signals for the plurality of three-dimensional hydrogel networks have a coefficient of variation of less than 25%.
233. The plurality of three-dimensional networks of embodiment 232, wherein the coefficient of variation is less than 20%.
234. The plurality of three-dimensional networks of embodiment 232, wherein the coefficient of variation is less than 15%.
235. The plurality of three-dimensional networks of embodiment 232, wherein the coefficient of variation is less than 10%.
236. The plurality of three-dimensional networks of embodiment 232, wherein the coefficient of variation is less than 9%.
237. The plurality of three-dimensional networks of embodiment 232, wherein the coefficient of variation is less than 8%.
238. The plurality of three-dimensional networks of embodiment 232, wherein the coefficient of variation is less than 7%.
239. The plurality of three-dimensional networks of embodiment 232, wherein the coefficient of variation is less than 6%.
240. The plurality of three-dimensional networks of embodiment 232, wherein the coefficient of variation is less than 5%.
241. The plurality of three-dimensional networks of any one of embodiments 232 to 240, wherein the coefficient of variation is at least 1%.
242. The plurality of three-dimensional networks of any one of embodiments 232 to 240, wherein the coefficient of variation is at least 2%.
243. The plurality of three-dimensional networks of any one of embodiments 232 to 239, wherein the coefficient of variation is at least 5%.
244. The plurality of three-dimensional networks of any one of embodiments 232 to 234, wherein the coefficient of variation is at least 10%.
245. A plurality of three-dimensional hydrogel networks each having a surface and an interior comprising (a) a cross-linked polymer, (b) one or more channels, and (c) fluorescent probe molecules immobilized in the network, wherein when exciting the fluorescent probe molecules to produce a signal, the measured signals for the plurality of three-dimensional hydrogel networks have a coefficient of variation of less than 25%.
246. The plurality of three-dimensional networks of embodiment 245, wherein the coefficient of variation is less than 20%.
247. The plurality of three-dimensional networks of embodiment 245, wherein the coefficient of variation is less than 15%.
248. The plurality of three-dimensional networks of embodiment 245, wherein the coefficient of variation is less than 10%.
249. The plurality of three-dimensional networks of embodiment 245, wherein the coefficient of variation is less than 9%.
250. The plurality of three-dimensional networks of embodiment 245, wherein the coefficient of variation is less than 8%.
251. The plurality of three-dimensional networks of embodiment 245, wherein the coefficient of variation is less than 7%.
252. The plurality of three-dimensional networks of embodiment 245, wherein the coefficient of variation is less than 6%.
253. The plurality of three-dimensional networks of embodiment 245, wherein the coefficient of variation is less than 5%.
254. The plurality of three-dimensional networks of any one of embodiments 245 to 253, wherein the coefficient of variation is at least 1%.
255. The plurality of three-dimensional networks of any one of embodiments 245 to 253, wherein the coefficient of variation is at least 2%.
256. The plurality of three-dimensional networks of any one of embodiments 245 to 252, wherein the coefficient of variation is at least 5%.
257. The plurality of three-dimensional networks of any one of embodiments 245 to 247, wherein the coefficient of variation is at least 10%.
258. The plurality of three-dimensional network of any one of embodiments 245 to 257, which is a plurality of three-dimensional networks according to any one of embodiments 232 to 257.
259. The plurality of three-dimensional networks of any one of embodiments 232 to 258, wherein the probe molecules are oligonucleotide probes.
260. The plurality of three-dimensional networks of any one of embodiments 232 to 259, wherein each three-dimensional network comprises at least 5 channels.
261. The plurality of three-dimensional networks of any one of embodiments 232 to 259, wherein each three-dimensional network comprises at least 10 channels.
262. The plurality of three-dimensional networks of any one of embodiments 232 to 261, wherein each three-dimensional network comprises a plurality of channels that converge at a point in the interior of the network such that the lateral distance between the channels decreases from the surface toward the point in the interior.
263. The plurality of three-dimensional networks of any one of embodiments 232 to 262, comprising long channels.
264. The plurality of three-dimensional networks of any one of embodiments 232 to 262, comprising long and short channels.
265. The plurality of three-dimensional networks of any one of embodiments 232 to 264, wherein the three-dimensional networks are positioned on an array.
266. The plurality of three-dimensional networks of any one of embodiments 232 to 264, wherein the three-dimensional networks are positioned on separate arrays.
267. The plurality of three-dimensional networks of any one of embodiments 232 to 265, comprising at least 2, at least 5, at least 10, at least 20, at least 100 and/or up to 1000 individual three-dimensional networks.
268. An array comprising a plurality of three-dimensional hydrogel networks according to embodiment 231 on a substrate.
269. An array comprising the plurality of three-dimensional hydrogel networks according to any one of embodiments 232 to 267 on a substrate.
270. An array produced or obtainable by the process of any one of embodiments 222 to 230.
271. The array of any one of embodiments 268 to 270, which comprises at least 8 three-dimensional hydrogel networks.
272. The array of any one of embodiments 268 to 270, which comprises at least 16 three-dimensional hydrogel networks.
273. The array of any one of embodiments 268 to 270, which comprises at least 24 three-dimensional hydrogel networks.
274. The array of any one of embodiments 268 to 270, which comprises at least 48 three-dimensional hydrogel networks.
275. The array of any one of embodiments 268 to 270, which comprises at least 96 three-dimensional hydrogel networks.
276. The array of any one of embodiments 268 to 270, which comprises at least 128 three-dimensional hydrogel networks.
277. The array of any one of embodiments 268 to 270, which comprises at least 256 three-dimensional hydrogel networks.
278. The array of any one of embodiments 268 to 270, which comprises at least 512 three-dimensional hydrogel networks.
279. The array of any one of embodiments 268 to 270, which comprises at least 1024 three-dimensional hydrogel networks.
280. The array of any one of embodiments 268 to 270, which comprises 24 to 8192 three-dimensional hydrogel networks.
281. The array of any one of embodiments 268 to 270, which comprises 24 to 4096 three-dimensional hydrogel networks.
282. The array of any one of embodiments 268 to 270, which comprises 24 to 2048 three-dimensional hydrogel networks.
283. The array of any one of embodiments 268 to 270, which comprises 24 to 1024 three-dimensional hydrogel networks.
284. The array of any one of embodiments 268 to 270, which comprises 24 three-dimensional hydrogel networks.
285. The array of any one of embodiments 268 to 270, which comprises 48 three-dimensional hydrogel networks.
286. The array of any one of embodiments 268 to 270, which comprises 96 three-dimensional hydrogel networks.
287. The array of any one of embodiments 268 to 270, which comprises 128 three-dimensional hydrogel networks.
288. T The array of any one of embodiments 268 to 270, which comprises 256 three-dimensional hydrogel networks.
289. The array of any one of embodiments 268 to 270, which comprises 512 three-dimensional hydrogel networks.
290. The array of any one of embodiments 268 to 270, which comprises 1024 three-dimensional hydrogel networks.
291. The array of any one of embodiments 268 to 290, wherein the three-dimensional hydrogel networks comprise probe molecules, and two or more of three-dimensional hydrogel networks comprise different species of probe molecules.
292. The array of any one of embodiments 268 to 291, wherein the three-dimensional hydrogel networks comprise probe molecules, and two or more three-dimensional hydrogel networks comprise the same species of probe molecules.
293. The array of any one of embodiments 268 to 290, wherein the three-dimensional hydrogel networks comprise probe molecules, and each of the three-dimensional hydrogel networks comprise the same species of probe molecules.
294. The array of any one of embodiments 268 to 293, wherein the plurality of three-dimensional hydrogel networks comprises one or more three-dimensional hydrogel networks comprising labeled control probe molecules.
295. The array of embodiment 294, wherein the labeled control probe molecules are fluorescently labeled.
296. The array of any one of embodiments 268 to 295, wherein the substrate comprises a microwell plate and each well of the microwell plate contains no more than a single three-dimensional hydrogel network.
297. A method for determining whether an analyte is present in a sample, comprising:
   (a) contacting a three-dimensional hydrogel network according to embodiment 231 or an array of any one of embodiments to 268 to 296 comprising probe molecules that are capable of binding to the analyte with the sample; and
   (b) detecting binding of the analyte to the probe molecules in the three-dimensional hydrogel network or array, thereby determining whether the analyte is present in the sample.
298. The method of embodiment 297, which further comprises washing the network or array comprising probe molecules between steps (a) and (b).
299. The method of embodiment 297 or embodiment 298, which further comprises contacting the network or array comprising probe molecules with a blocking reagent prior to step (a).
300. The method of any one of embodiments 297 to 299, further comprising quantifying the amount of analyte bound to the three-dimensional hydrogel network or array comprising probe molecules.
301. A method for determining whether an analyte is present in each sample in a plurality of samples, comprising:
   (a) contacting an array of any one of embodiments 268 to 296 comprising probe molecules that are capable of binding to the analyte with the samples; and
   (b) detecting binding of the analyte to the probe molecules in the array, thereby determining whether the analyte is present in each sample in the plurality of samples.
302. A method for determining whether an analyte is present in each sample in a plurality of samples, comprising:
   (a) contacting an array of any one of embodiments 268 to 296 comprising probe molecules that are capable of binding to the analyte with the samples and comprising control probe molecules, wherein the array has been used and washed prior to step (a); and
   (b) detecting binding of the analyte to the probe molecules in the array, thereby determining whether the analyte is present in each sample in the plurality of samples.
303. A method for determining whether more than one species of analyte is present in a sample, comprising:
   (a) contacting an array of any one of embodiments 268 to 296 comprising different species of probe molecules that are capable of binding to the different species of analytes with the sample; and
   (b) detecting binding of the analytes to the probe molecules in the array, thereby determining whether more than one species of analyte are present in the sample.
304. A method for determining whether more than one species of analyte is present in a sample, comprising:
   (a) contacting an array of any one of embodiments 268 to 296 comprising different species of probe molecules that are capable of binding to the different species of analytes with the sample and comprising control probe molecules, wherein the array has been used and washed prior to step (a); and
   (b) detecting binding of the analytes to the probe molecules in the array, thereby determining whether more than one species of analyte are present in the sample.
305. The method of any one of embodiments 301 to 304, in which:
   (a) the substrate of the array comprises a microwell plate;
   (b) each well of the microwell plate contains no more than a single three-dimensional hydrogel network; and
   (c) contacting the array with the samples comprises contacting each well with no more than a single sample.
306. The method of any one of embodiments 301 to 305, which further comprises washing the array comprising probe molecules between steps (a) and (b).
307. The method of any one of embodiments 301 to 306, which further comprises contacting the array comprising probe molecules with a blocking reagent prior to step (a).
308. The method of any one of embodiments 301 to 307, further comprising quantifying the amount of analyte or analytes bound to the array.
309. The method of any one of embodiments 297 to 308, further comprising reusing the array.
310. The method of embodiment 309, wherein the array is reused at least 5 times.
311. The method of embodiment 309, wherein the array is reused at least 10 times.
312. The method of embodiment 309, wherein the array is reused at least 20 times.
313. The method of embodiment 309, wherein the array is reused at least 30 times.
314. The method of embodiment 309, wherein the array is reused at least 40 times.
315. The method of embodiment 309, wherein the array is reused at least 50 times.
316. The method of embodiment 310, which comprises reusing the array 5 to 20 times.
317. The method of embodiment 310, which comprises reusing the array 5 to 30 times.
318. The method of embodiment 310, which comprises reusing the array 10 to 50 times.
319. The method of embodiment 310, which comprises reusing the array 10 to 20 times.
320. The method of embodiment 310, which comprises reusing the array 10 to 30 times.
321. The method of embodiment 310, which comprises reusing the array 20 to 40 times.
322. The method of embodiment 310, which comprises reusing the array 40 to 50 times.
323. The method of any one of embodiments 309 to 322, which comprises washing the array between reuses.
324. The method of embodiment 323, wherein the array is washed under denaturing conditions.
325. The method of embodiment 324, wherein the denaturing conditions comprise exposing the array to heat.
326. The method of embodiment 324, wherein the denaturing conditions comprise exposing the array to low salt concentrations.
327. The method of embodiment 324, wherein the denaturing conditions comprise exposing the array to both heat and low salt concentrations.
328. The method of embodiment 324, wherein the denaturing conditions are removed prior to reuse.
329. The method of embodiment 328, wherein the denaturing conditions comprise exposing the array to heat and wherein the temperature is lowered prior to reuse.
330. The method of embodiment 328, wherein the denaturing conditions comprise exposing the array to low salt concentrations and wherein the salt concentration is increased prior to reuse.
331. The method of embodiment 328, wherein the denaturing conditions comprise exposing the array to both heat and low salt concentrations and wherein the temperature is lowered and the salt concentration is increased prior to reuse.
332. The method of any one of embodiments 309 to 331, wherein the array comprises at least one three-dimensional hydrogel network comprising a fluorescently labelled oligonucleotide as a reusability control.
333. The method of embodiment 332, which comprises testing the fluorescent signal strength.
334. The method of embodiment 333, wherein the reusability control retains at least
70% of its initial fluorescence signal strength after 10 uses.
335. The method of embodiment 334, wherein the reusability control retains at least
50% of its signal strength after 20 uses.
336. The method of any one of embodiments 332 to 335, wherein the array is no longer reused after the reusability control loses more than 50% of its signal strength.
337. The method of any one of embodiments 297 to 336, wherein analyte is a nucleic acid.
338. The method of embodiment 337, wherein the nucleic acid is a polymerase chain reaction (PCR) amplicon.
339. The method of embodiment 337, wherein the PCR amplicon is amplified from a biological sample or environmental sample.
340. The method of embodiment 339, wherein the PCR amplicon is amplified from a biological sample.
341. The method of embodiment 339, wherein the PCR amplicon is amplified from an environmental sample.
342. The method of embodiment 340, wherein the biological sample is a blood, serum, plasma, tissue, cells, saliva, sputum, urine, cerebrospinal fluid, pleural fluid, milk, tears, stool, sweat, semen, whole cells, cell constituent, cell smear, or an extract or derivative thereof.
343. The method of embodiment 342, wherein the biological sample is mammalian blood, serum or plasma or an extract thereof.
344. The method of embodiment 343, wherein the biological sample is human or bovine blood, serum or plasma or an extract thereof.
345. The method of embodiment 342, wherein the biological sample is milk or an extract thereof.
346. The method of embodiment 345, wherein the biological sample is cow's milk or an extract thereof.
347. The method of any one of embodiments 337 to 346, wherein nucleic acid is labeled.
348. The method of embodiment 347, wherein the nucleic acid is fluorescently labeled.
349. A kit comprising
   (a) a mixture comprising:
      (i) an aqueous salt solution;
      (ii) a water-soluble polymer;
      (iii) cross-linker moieties, which are optionally covalently attached to the water-soluble polymer; and
      (iv) optionally, probe molecules; and
   (b) a substrate;
   wherein the concentration of the water-soluble polymer chains in the mixture is below the saturation concentration of the water-soluble polymer chains, and optionally less than 1 mg/ml, and the mixture comprises phosphate ions at a concentration ranging from 125 mM to less than 350 mM.
350. The kit of embodiment 349, wherein the concentration of the water-soluble polymer in the mixture is 0.1 mg/ml and the concentration of phosphate in the mixture is 250 mM.
351. The kit of any one of embodiments 349 to 350, wherein the water-soluble polymer chains comprise a polymer polymerized from dimethylacrylamide (DMAA), methacryloyloxybenzophenone (MABP), and sodium 4-vinylbenzenesulfonate (SSNa), optionally in a DMAA:MABP:SSNa molar ratio of 92.5:5:2.5.

While various specific embodiments have been illustrated and described, it will be appreciated that various changes can be made without departing from the spirit and scope of the disclosure(s).

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A process for making a three-dimensional hydrogel network, comprising:
(a) exposing a mixture (optionally positioned on the surface of a substrate) to salt crystal forming conditions, the mixture comprising:
(v) an aqueous salt solution,
(vi) water-soluble polymer chains,
(vii) cross-linker moieties, and
(viii) optionally, probe molecules,
thereby forming a mixture containing one or more salt crystals;
(b) cross-linking the water-soluble polymer chains in the mixture containing one or more salt crystals, thereby forming a hydrogel containing one or more salt crystals; and
(c) contacting the hydrogel containing one or more salt crystals with a solvent in which the one or more salt crystals are soluble, thereby dissolving the salt crystals;
thereby forming the three-dimensional hydrogel network,
wherein:
A) the concentration of the water-soluble polymer chains in the mixture of step (a) is such that precipitation of water-soluble polymer chains from the mixture does not occur before formation of the one or more salt crystals in step (a); and/or
B) the concentration of the water-soluble polymer chains in the mixture of step (a) is such that the water-soluble polymer chains and salt crystals co-precipitate in step (a); and/or
C) the concentration of the water-soluble polymer chains in the mixture of step (a) is less than 1 mg/ml.

2. The process of claim 1, wherein the concentration of the water-soluble polymer chains in the mixture of step (a) is such that precipitation of water-soluble polymer chains from the mixture does not occur before formation of the one or more salt crystals in step (a).

3. The process of claim 1 or claim 2, wherein the concentration of the water-soluble polymer chains in the mixture of step (a) is such that the water-soluble polymer chains and salt crystals co-precipitate during step (a).

4. The process of any one of claims 1 to 3, wherein the concentration of the water-soluble polymer chains in the mixture of step (a) is less than 1 mg/ml.

5. The process of any one of claims 1 to 4, wherein the concentration of the water-soluble polymer chains in the mixture of step (a) ranges from a lower limit (“polymer lower limit”) that is at least 0.01 mg/ml to an upper limit (“polymer upper limit”) that is less than 1 mg/ml.

6. The process of claim 5, wherein the polymer lower limit is 0.01 mg/ml, 0.05 mg/ml, or 0.1 mg/ml and/or the polymer upper limit is 0.5 mg/ml, 0.4 mg/ml, 0.3 mg/ml, 0.2 mg/ml, or 0.1 mg/ml, optionally wherein the concentration of the water-soluble polymer chains in the mixture of step (a) is 0.1 mg/ml.

7. The process of any one of claims 1 to 6, wherein the cross-linker moieties are photoreactive and step (b) comprises cross-linking the water-soluble polymer chains with a UV light energy dose of less than 1 J/cm².

8. The process of claim 7, wherein step (b) comprises cross-linking the water-soluble polymer chains with a UV light energy dose that ranges from a lower limit (“cross-linking energy lower limit”) that is at least 0.4 J/cm² to an upper limit (“cross-linking energy upper limit”) that is less than 1 J/cm².

9. The process of claim 8, wherein the cross-linking energy lower limit is 0.4 J/cm²
0.5 J/cm², 0.6 J/cm², or 0.7 J/cm² and/or the cross-linking energy upper limit is 0.9 J/cm², 0.8 J/cm², or 0.7 J/cm², optionally wherein step (b) comprises cross-linking the water-soluble polymer chains with a UV energy dose of 0.7 J/cm².

10. The process of any one of claims 1 to 9, wherein the aqueous salt solution comprises phosphate ions.

11. The process of claim 10, wherein the concentration of the phosphate ions in the mixture of step (a) ranges from a lower limit (“phosphate lower limit”) that is at least 125 mM to an upper limit (“phosphate upper limit”) that is less than 350 mM.

12. The process of claim 11, wherein the phosphate lower limit is 125 mM, 150 mM,
200 mM, or 225 mM and/or the phosphate upper limit is 340 mM, 300 mM, or 250 mM, optionally wherein the concentration of phosphate ions in the mixture of step (a) is 250 mM.

13. The process of any one of claims 1 to 12, wherein the aqueous salt solution comprises a sodium phosphate solution.

14. The process of any one of claims 1 to 13, wherein the salt crystal forming conditions result in formation one or more needle-shaped crystals such that one or more long channels are produced after dissolution of the salt crystals.

15. The process of any one of claims 1 to 9, wherein the mixture of step (a) comprises at least two types of monovalent metal ions having a total concentration of at least
500 mM, optionally wherein the mixture of step (a) comprises at least two types of monovalent metal ions having a total concentration of 500 mM to 1000 mM.

16. The process of claim 15, wherein the salt crystal forming conditions result in formation one or more needle-shaped crystals such that one or more long channels are produced after dissolution of the salt crystals.

17. The process of any one of claim 15 or claim 16, wherein the salt crystal forming conditions result in formation of one or more compact crystals such that one or more short channels are produced after dissolution of the salt crystals.

18. The process of any one of claims 1 to 17, wherein the water-soluble polymer comprises a polymer polymerized from dimethylacrylamide (DMAA), methacryloyloxybenzophenone (MABP), and sodium 4-vinylbenzenesulfonate (SSNa), optionally comprising 2.5 to 7.5 mol % MABP, 2 to 5 mol % SSNa, and the balance DMAA.

19. A process for making a three-dimensional hydrogel network, comprising:
(a) exposing a mixture (optionally positioned on the surface of a substrate) to salt crystal forming conditions, the mixture comprising:
(iv) an aqueous salt solution comprising phosphate ions at a concentration ranging from a lower limit (“phosphate lower limit”) that is at least 125 mM to an upper limit (“phosphate upper limit”) that is less than 350 mM
(v) water-soluble polymer chains at a concentration of less than 1 mg/ml, wherein the water-soluble polymer chains comprise a polymer polymerized from dimethylacrylamide (DMAA), methacryloyloxybenzophenone (MABP), and sodium 4-vinylbenzenesulfonate (SSNa), and
(vi) optionally, probe molecules,
thereby forming a mixture containing one or more salt crystals;
(b) cross-linking the water-soluble polymer chains in the mixture containing one or more salt crystals, thereby forming a hydrogel containing one or more salt crystals; and
(c) contacting the hydrogel containing one or more salt crystals with a solvent in which the one or more salt crystals are soluble, thereby dissolving the salt crystals;
thereby forming the three-dimensional hydrogel network.

20. The process of claim 19, wherein the concentration of the water-soluble polymer chains in the mixture of step (a) ranges from a lower limit (“polymer lower limit”) that is at least
0.01 mg/ml to an upper limit (“polymer upper limit”) that is less than 1 mg/ml.

21. The process of claim 20, wherein the polymer lower limit is 0.01 mg/ml, 0.05 mg/ml, or 0.1 mg/ml and/or the polymer upper limit is 0.5 mg/ml, 0.4 mg/ml, 0.3 mg/ml, 0.2 mg/ml, or 0.1 mg/ml.

22. The process of claim 19, wherein the concentration of the water-soluble polymer chains in the mixture of step (a) is 0.1 mg/ml.

23. The process of any one of claims 19 to 22, wherein the phosphate lower limit is
125 mM, 150 mM, 200 mM, or 225 mM and/or the phosphate upper limit is 340 mM, 300 mM, or 250 mM.

24. The process of any one of claims 19 to 22, wherein the concentration of phosphate ions in the mixture of step (a) is 250 mM.

25. The process of any one of claims 19 to 24, wherein the aqueous salt solution comprises a sodium phosphate solution.

26. The process of any one of claims 19 to 25, wherein step (b) comprises cross-linking the water-soluble polymer chains with a UV light energy dose of less than 1 J/cm².

27. The process of claim 26, wherein step (b) comprises cross-linking the water-soluble polymer chains with a UV light energy dose that ranges from a lower limit (“cross-linking energy lower limit”) that is at least 0.4 J/cm² to an upper limit (“cross-linking energy upper limit”) that is less than 1 J/cm².

28. The process of claim 27, wherein the cross-linking energy lower limit is 0.4 J/cm², 0.5 J/cm², 0.6 J/cm², or 0.7 J/cm², and/or the cross-linking energy upper limit is 0.9 J/cm², 0.8 J/cm², or 0.7 J/cm².

29. The process of claim 26, wherein step (b) comprises cross-linking the water-soluble polymer chains with a UV energy dose of 0.7 J/cm².

30. The process of any one of claims 19 to 29, wherein the water-soluble polymer comprises a polymer polymerized from dimethylacrylamide (DMAA), methacryloyloxybenzophenone (MABP), and sodium 4-vinylbenzenesulfonate (SSNa) and comprising 2.5 to 7.5 mol % MABP, 2 to 5 mol % SSNa, and the balance DMAA.

31. The process of any one of claims 19 to 29, wherein the water-soluble polymer comprises a polymer polymerized from dimethylacrylamide (DMAA), methacryloyloxybenzophenone (MABP), and sodium 4-vinylbenzenesulfonate (SSNa) in a DMAA:MABP:SSNa molar ratio of 92.5:5:2.5.

32. The process of any one of claims 1 to 31, wherein the salt crystal forming conditions comprise dehydrating the mixture, optionally by heating the mixture, exposing the mixture to a vacuum, reducing the humidity of the atmosphere surrounding the mixture, or a combination thereof.

33. The process of any one of claims 1 to 32, wherein the salt crystal forming conditions comprise cooling the mixture until the mixture becomes supersaturated with a salt, optionally by contacting the mixture with a gas that has a temperature which is lower than the temperature of the mixture.

34. The process of any one of claims 1 to 33, wherein the mixture of step (a) further comprises probe molecules.

35. The process of claim 34, wherein at least some, the majority or all the probe molecules comprise a nucleic acid.

36. The process of claim 35, wherein the nucleic acid is an oligonucleotide.

37. The process of claim 36, wherein the concentration of the oligonucleotide in the mixture of step (a) ranges from 5 μM to 35 μM.

38. The process of any one of claims 1 to 37, further comprising, prior to step (a), a step of applying the mixture to a surface of a substrate.

39. The process of claim 38, wherein the mixture is applied in a volume of 100 pl to
5 nl.

40. A process for making an array, comprising generating a plurality of three-dimensional hydrogel networks by the process of any one of claims 1 to 39 at discrete spots on the surface of the same substrate.

41. A process for making an array, comprising positioning a plurality of three-dimensional hydrogel networks produced or obtainable according to the process of any one of claims 1 to 39 at discrete spots on a surface of the same substrate.

42. A three-dimensional hydrogel network produced or obtainable by the process of any one of claims 1 to 39.

43. A plurality of three-dimensional hydrogel networks each having a surface and an interior comprising (a) a cross-linked polymer, (b) one or more channels, and (c) probe molecules immobilized in the network, wherein when contacted with an analyte capable of binding to the probe molecules to produce a signal, the measured signals for the plurality of three-dimensional hydrogel networks have a coefficient of variation of less than 25%.

44. The plurality of three-dimensional networks of claim 43, wherein the coefficient of variation is less than 20%, less than 15%, less than 10%, less than 9%, less than 8%, less than
7%, less than 6%, or less than 5%, and/or at least 1% or at least 2%.

45. The plurality of three-dimensional networks of claim 43 or claim 44, wherein each three-dimensional network comprises a plurality of channels that converge at a point in the interior of the network such that the lateral distance between the channels decreases from the surface toward the point in the interior.

46. An array comprising a plurality of three-dimensional hydrogel networks according to claim 42 on a substrate.

47. An array comprising the plurality of three-dimensional hydrogel networks according to any one of claims 43 to 45 on a substrate.

48. An array produced or obtainable by the process of claim 40 or claim 41.

49. A method for determining whether an analyte is present in a sample, comprising:
(a) contacting a three-dimensional hydrogel network according to claim 42 or an array of any one of claims to 46 to 48 comprising probe molecules that are capable of binding to the analyte with the sample; and
(b) detecting binding of the analyte to the probe molecules in the three-dimensional hydrogel network or array, thereby determining whether the analyte is present in the sample.

50. A kit comprising
(a) a mixture comprising:
(i) an aqueous salt solution;
(ii) a water-soluble polymer;
(iii) cross-linker moieties, which are optionally covalently attached to the water-soluble polymer; and
(iv) optionally, probe molecules; and
(b) a substrate;
wherein the concentration of the water-soluble polymer chains in the mixture is less than 1 mg/ml, and the mixture comprises phosphate ions at a concentration ranging from 125 mM to less than 350 mM.

51. The kit of claim 50, wherein the concentration of the water-soluble polymer in the mixture is 0.1 mg/ml and the concentration of phosphate in the mixture is 250 mM.

52. The kit of claim 50 or claim 51, wherein the water-soluble polymer chains comprise a polymer polymerized from dimethylacrylamide (DMAA), methacryloyloxybenzophenone (MABP), and sodium 4-vinylbenzenesulfonate (SSNa), optionally in a DMAA:MABP:SSNa molar ratio of 92.5:5:2.5.
